(19) United States
(12) Patent Application Publication
(10) Pub. No.: US 2025/0388861 A1
(43) Pub. Date: Dec. 25, 2025

(54) DIRECTIONALLY-SPECIFIC MAGNETIC MODULATION OF THE CELL SECRETOME FOR MEDICAL AND COMMERCIAL APPLICATIONS

(71) Applicant: National University of Singapore, Singapore (SG)

(72) Inventors: Alfredo Franco-Obregon, Singapore (SG); Yee Kit Tai, Singapore (SG); Vinicius Rosa, Singapore (SG); Rufaihah Binte Adbul Jalil, Singapore (SG); Marek Kukumberg, Singapore (SG); Jun Kit Craig Wong, Singapore (SG)

(73) Assignee: National University of Singapore, Singapore (SG)

(21) Appl. No.: 18/879,650

(22) PCT Filed: Jun. 28, 2023
(86) PCT No.: PCT/SG2023/050453

§ 371 Date: Dec. 27, 2024

(30) Foreign Application Priority Data
Jun. 28, 2022 (SG) 10202250309E

(51) Int. Cl. C12N 5/077; A23L 13/00

(52) U.S. Cl. CPC C12N 5/0658; A23L 13/00; C12N 2529/00

(57) ABSTRACT

The present invention generally relates to the provision of a conditioned medium comprising magnetically-induced cell secretome, the methods of producing the same, and a system for the production of said conditioned medium. More particularly, the present invention provides an improved method of producing a conditioned medium comprising cell secretome induced by a directionally-specific pulsing electromagnetic field (PEMF), wherein the conditioned medium of the present invention is capable of enhancing proliferation, differentiation, survival or senescence of recipient cells. Also provided are a system for the production of said PEMF-conditioned medium and the improved conditioned medium thereof, suitable for use in medical and commercial applications.

### FIELD OF THE INVENTION

The present invention generally relates to the provision of a conditioned medium comprising magnetically-induced cell secretome, methods of producing the same, and a system for the production of said conditioned medium. More particularly, the present invention provides an improved method of producing a conditioned medium comprising cell secretome induced by a directionally-specific pulsing electromagnetic field (PEMF), wherein the conditioned medium of the present invention is capable of enhancing proliferation, differentiation, survival or senescence of recipient progenitor and/or stem cells. Also provided are a system for the production of said PEMF-conditioned medium and the improved conditioned medium thereof, suitable for use in medical and commercial applications.

### BACKGROUND OF THE INVENTION

Cells communicate by virtue of their secretomes. The secretome consists of paracrine, autocrine, and endocrine soluble factors as well as extracellular vesicles that are released from cells to govern tissue development, regeneration, metabolic balance, systemic immunity and cross-talk within and between tissues.

On the organismal level, the muscle secretome reigns supreme. The skeletal muscle, being the largest tissue mass, has evolved to play a fundamental role in systemic regeneration and metabolic balance. This aspect of muscle function is largely mediated via the actions of its secretome, which acts locally (muscle) as well as systemically (i.e., on other body tissues). The muscle secretome consists of a myriad of regenerative, metabolic, anti-inflammatory and immunity-boosting factors released into the systemic circulation as either individual or vesicle-encapsulated components.

In response to energy metabolism, such as that required to initiate and sustain exercise, muscle releases the contents of its secretome into the bloodstream for systemic delivery. In this regard, muscle upregulates the production and release of blood-borne soluble factors collectively known as myokines. PGC-1α-dependent transcriptional co-activation of the genes involved in mitochondrial homeostasis (Li, J. et al., Antioxidants (Basel) 9 (2020); Louzada, R. A. et al., Antioxid Redox Signal (2020)) instigate the myokine response (Ost, M. et al., Free Radic Biol Med 98, 78-89 (2016); Scheele, C., Nielsen, S., and Pedersen, B. K., Trends Endocrinol Metab 20, 95-99 (2009)), whereas extracellular calcium entry (Hao, Y. et al., Journal of Controlled Release 340, 136-148 (2021)) as well as mitochondrial respiration (Louzada, R. A. et al., Antioxid Redox Signal (2020)) and exercise (Vechetti Jr, I. J. et al., The FASEB Journal 35, e21644 (2021)) stimulate muscular extracellular vesicle (EV) release. These two aspects of the secretome are not mutually exclusive, but are activated in parallel by transduction pathways activated by exercise that are common to both limbs of the response (Louzada, R. A. et al., Antioxid Redox Signal (2020); Li, G. et al., Journal of cellular biochemistry 120, 14262-142739 (2019)).

Apart from muscle secretomes, the secretomes of stem cells are also known to promote tissue differentiation and development. Despite the obvious importance of the cell secretome, methods of controlling its release safely and effectively in vitro and ex vivo are not available. The constitutive release is slow and inefficient and is often not adequate to produce the desired response. Therefore, researchers often revert to overgrowing cells in order to collect sufficient secretome, which holds inherent problems. Overgrowth of cells promotes senescence and the production of a secretome that likewise promotes senescence (Senescence-Associated Secretory Phenotype (SASP)). Further, the cell secretome has been shown to be stage-specific (i.e., the cell secretome mirrors the status of the cell). Cells during log phase expansion produce a secretome that promotes proliferation, whereas cells undergoing differentiation produce a secretome that forestalls proliferation at the expense of differentiation. Thus, cell overgrowth also runs the risk of generating cells from distinct stages of in vitro differentiation.

On the other hand, while genetic modification or drugs can enhance and promote secretome release, they however may pose good manufacturing practice (GMP) barriers and regulatory hurdles, and ultimately slow down translation and its acceptance. Commercial and academic attempts to recapitulate a desired effect of the cell secretome via the supplementation with exogenous agents are costly and yet rely, to a large degree, on guesswork. In this regard, a potentially effective approach is to allow the cell to produce what it needs with biophysical induction.

Pulsing electromagnetic fields (PEMFs) have been shown to stimulate myogenesis and mitochondrial respiration in cells (Yap, J. L. Y. et al., Faseb j 33, 12853-12872 (2019)) and in mice (Tai, Y. K. et al., Faseb j 34, 11143-11167 (2020)). Magnetic enhancements of both in vitro and in vivo myogenesis were associated with PGC-1a transcriptional co-activation of mitochondriogenesis and mitohormetic survival adaptations. A key player in these magnetic mitohormetic responses was the Transient Receptor Potential Canonical 1 (TRPC1) calcium-permeable channel whose expression and function were required to elicit magnetically-stimulated chondrogenesis (Parate, D. et al., Sci Rep 7, 9421 (2017)), neurogenesis (Madanagopal, T. T. et al., Eur Cell Mater 41, 216-232 (2021)) and myogenesis (Yap, J. L. Y. et al., Faseb j 33, 12853-12872 (2019); Tai, Y. K. et al., Faseb j 34, 11143-11167 (2020)). TRPC1 reintroduction was also shown to be necessary and sufficient to reinstate magnetically-induced mitochondrial respiration and enhanced myogenesis in a CRISPR/Cas9 TRPC1-knockdown skeletal muscle cell line (Kurth, F. et al., Biosyst 4, e2000146)). Analogous magnetic stimulation was capable of activating the secretome response of mesenchymal stem cells to promote in vitro chondrogenesis and improve survival following induced inflammation (Parate, D. et al., Stem Cell Res Ther 11, 46 (2020)) in association with TRPC1 expression ((Parate, D. et al., Sci Rep 7, 9421 (2017)). Given the accepted interdependency between mitochondrial respiration and secretome response, PEMF stimulation, via its effects on the TRPC1 channel signalling, might hence represent a viable approach to improve and optimise the production of cell secretome for clinical, commercial and other applications.

Accordingly, there is a need to provide improved methods of producing cell secretome and conditioned media comprising the same that overcome or at least ameliorate, one or more of the drawbacks described above.

### SUMMARY OF THE INVENTION

The present invention relates to the use of a directionally-specific PEMF induction paradigm to produce a conditioned medium comprising a magnetically-induced secretome of interest. Disclosed herein are methods of producing said conditioned medium capable of promoting proliferation, differentiation or senescence of progenitor and/or stem cells, a method of proliferating and differentiating progenitor and/or stem cells, a system for the production thereof, and a PEMF-conditioned medium thereof.

In a first aspect, there is provided a method of producing a conditioned medium capable of promoting proliferation, differentiation or senescence of progenitor and/or stem cells, wherein the method comprises the steps:
a) culturing proliferating, differentiating or senescent (oxidatively stressed) progenitor and/or stem cells in media as a suspension culture in a bioreactor;
b) exposing the proliferating, differentiating, or senescent (oxidatively stressed) progenitor and/or stem cells to downward-directed or upward-directed low amplitude pulsed electromagnetic fields (PEMFs); and
c) collecting the PEMF-conditioned media (pCM) which comprises a secretome that reflects the status of the cells in the bioreactor and has proliferation-promoting capability, cell survival-promoting capability, cell differentiation-promoting capability, or cell senescence-promoting capability.

In a second aspect, there is provided a method of proliferating or differentiating progenitor and/or stem cells, comprising adding the pCM from proliferating or differentiating cells, respectively, as defined in the first aspect, to a progenitor cell culture.

In a third aspect, there is provided a system comprising:
i) a bioreactor within which a first culture of progenitor and/or stem cells in suspension is subjected to downward-directed low amplitude pulsed electromagnetic fields (PEMFs), or upward-directed low amplitude pulsed electromagnetic fields (PEMFs), to produce a PEMF-conditioned media (pCM), and
ii) a second cell culture to be expanded (via induced proliferation), differentiated, or induced into a senescent (oxidatively stressed) state, wherein said PEMF-conditioned media (pCM) from (i) is provided to cell culture (ii).

In a fourth aspect, there is provided a method of enhancing cultured meat production, comprising feeding a cell-based meat culture a pCM produced by the method of the first aspect.

In a fifth aspect, there is provided a PEMF-conditioned media (pCM), produced by the method of the first aspect, preferably comprising exosomes.

In a sixth aspect, there is provided a method of pre-conditioning proliferating, differentiating or senescent (oxidatively stressed) progenitor and/or stem cells for use in the production of a PEMF-conditioned media (pCM), wherein the method comprises contacting a sample of proliferating, differentiating or senescent (oxidatively stressed) progenitor and/or stem cells with a pCM according to the fifth aspect.

In contrast to the application of direct magnetic exposure, the pCM of the present disclosure does not trigger oxidative stress in recipient cells and is therefore capable of improving survival advantage of the recipient cells. In addition, the pCM obtained by the methods disclosed herein are also more effective at improving cellular survival compared to conventional fetal bovine serum and does not require the use of exogenous and expensive growth and/or trophic factors in the cultures. These and other advantages of the present disclosure will become readily apparent to those skilled in this art from the following detailed description.

### BRIEF DESCRIPTION OF THE FIGURES

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 shows that magnetic-induced proliferation enhancement can be conferred to naïve myoblasts with the provision of PEMF-conditioned media (pCM). A) Schematic depiction of the experimental paradigm for proliferation assessment under basal conditions (dark grey) or following magnetic stimulation (light grey) for myoblasts directly kept in their original bathing media (solid) or upon the transfer of the conditioned media to naïve age-matched recipient cultures (cross-hatched). B) Proliferation induction normalized (fold change) to the unexposed condition (solid dark grey), as indicated. Recipient cultures were given conditioned media (CM) either from magnetically stimulated (pCM) or unexposed (cCM) donor cultures, light grey and dark grey cross-hatched, respectively. Data represents the average of n=14 biological replicates. C) Magnetic-induced proliferation enhancement could be nullified by the replacement of pCM with cCM. Depiction of proliferation assessment under basal conditions (dark grey) or following magnetic stimulation (light grey), directly (solid) or upon transfer of cCM from unexposed age-matched sister cultures (cross-hatched). D) Quantification of induced proliferation expressed as fold change relative to direct 0 mT (dark grey). Hatched bars represent the values for previously exposed recipient 20 cultures after cCM washout. Data represents the average of n=7 biological replicates. E) Depiction of proliferation assessment for suspension cultures without magnetic exposure (dark grey) or upon magnetic stimulation (light grey), prior to the replating of the cells (dots) and their respective CMs into empty culture flasks (solid) or upon the delivery of the distinct CMs (without cells) to age-matched recipient cultures (hatched). F) Quantification of induced 25 proliferation expressed as fold change relative to direct 0 mT. Data represents the average of n=3 biological replicates. All magnetic exposures consisted of downward-directed PEMFs at an amplitude of 1.5 mT applied once for 10 min. Conditioned media was transferred to recipient cultures 6 h (A-B) or 30 min (E, F) following PEMF or sham exposure of donor cultures; media was removed from recipient cultures 1 h after PEMF or sham exposure and replaced age-matched media from untreated donor sister cultures (C-D). Error bars represent the standard error of the mean, with ^{#}p<0.0001, analyzed using One-Way ANOVA with Sidak's multiple comparisons test. All biological replicates were derived from the means of three technical replicates.

FIG. 2 shows that magnetic field directionality determines the efficacy of pCM. A) Schematic representation of culture flasks under basal conditions (unexposed; dark grey) or upon direct exposure to magnetic fields (light grey) of up, down or horizontal directionalities, as indicated. B) Quantification of induced proliferation in response to upward (vertical light grey stripes), downward (solid light grey) or horizontal (horizontal light grey stripes) field exposure relative to unexposed cultures (dark grey). Data represents the average of n=3-14 biological replicates. C) Depiction of recipient cultures receiving cCM (dark grey) or pCM (light grey) that had been generated under analogous exposure conditions as shown in A. D) Quantification of induced proliferation in recipient cultures in response to pCM generated from donor cultures receiving upward (vertical light grey stripes), downward (solid light grey) or horizontal (horizontal light grey stripes) field exposure relative to cultures receiving cCM (dark grey). Data presents the average of n=7-15 biological replicates. E) Schematic representation depicting cells in suspension under basal conditions (dark grey) or upon exposure to upward (vertical light grey stripes), downward (solid light grey), or horizontal (horizontal light grey stripes) magnetic fields before replating both the suspended cells (light grey dots) and their respective conditioned-media into new culture flasks for subsequent growth assessment. F) Quantification of induced proliferation in response to the indicated pCMs plus cells relative to cultures receiving cCM and unexposed cells. Data represents the average of n=3 biological replicates. G) Depiction of the transfer of CM (cCM or pCM) from cells in suspension to recipient cells prior to cell growth assessment. H) Quantification of induced proliferation in recipient cultures after receiving the indicated pCMs relative to cultures receiving cCM. Recipient cultures were either grown in their basal media (diagonal dark grey stripes), received cCM from unexposed suspension cells (solid dark grey), or pCM from suspension cells that were exposed to upward (vertical light grey stripes) or downward (solid light grey) magnetic fields. Data represents the average of n=9 biological replicates. All biological replicates were derived from the means of three technical replicates. All magnetic exposures consisted of PEMFs at an amplitude of 1.5 mT applied once for 10 min. Error bars represent the standard error of the mean, with *p<0.05, ** p<0.01, *** p<0.001 and ^{#}p<0.0001, analyzed using One-Way ANOVA with Dunnett's multiple comparisons test. “ns” indicates statistical nonsignificant differences. “None” refers to cultures given cCM or no magnetic exposure. “Up,” “Down” and “Hori,” refer to cultures that were exposed to 1.5 mT PEMFs in the upward, downward and horizontal direction, respectively.

FIG. 3 shows that aminoglycoside antibiotics block the ability of magnetic fields to stimulate proliferation. A) Depiction of recipient cultures receiving cCM (dark grey) or pCM (light grey) that had been generated with or without streptomycin added to the media of the donor cells at the time of downward field exposure (light grey) or no exposure (dark grey), as indicated. B) Bar chart showing the pooled data for recipient cell numbers expressed as fold change relative to recipient cells in unexposed cCM without streptomycin. Data represents the average of n=6 biological replicates. C) Depiction of recipient cultures receiving cCM (dark grey) or pCM (light grey) that had been generated from cells in suspension (either 1× or 5× cell density, with or without streptomycin added) under basal conditions (dark grey) or exposed to downward (solid light grey) or upward (vertical light grey stripes) magnetic fields. D) Bar chart showing the pooled data of induced proliferation from recipient cultures expressed as fold change relative to 1× cultures receiving unexposed cCM without streptomycin. Data represents the average of n=7-13 biological replicates. All biological replicates were derived from the means of three technical replicates. All magnetic exposures consisted of PEMFs at an amplitude of 1.5 mT applied once for 10 min. Error bars represent the standard error of the mean, with ** p<0.01, *** p<0.001, and ^{#}p<0.0001 analyzed using One-Way ANOVA with Sidak's multiple comparisons test. “None” refers to cultures given cCM or no magnetic exposure. “Up,” “Down” and “Hori,” refer to cultures that were exposed to 1.5 mT PEMFs in the upward, downward and horizontal direction, respectively.

FIG. 4 shows that direct exposure of myoblasts to downward field preferentially enhances TRPC channel, cell cycle progression and myogenic regulator expressions. Quantification of the relative protein expression (normalized to GAPDH) for A) TRPC1, B) TRPC3, C) TRPC6, D) TRPM7, E) Cyclin B1, F) Cyclin D1, G) p21, H) MyoD, I) MyoG and J) HTRA1 from cells directly exposed to downward (middle bar) or upward (right bar) magnetic fields expressed as fold change with respect to the unexposed condition (0 mT; left bar). Data represents the average of n=10-12 biological replicates. All magnetic exposures consisted of PEMFs at an amplitude of 1.5 mT applied once for 10 min. Protein expression was determined 24 h after direct exposure to PEMFs of the indicated characteristics. Error bars represent the standard error of the mean, with *p<0.05, ** p<0.01, and *** p<0.001, analyzed using One-Way ANOVA with Sidak's multiple comparisons test. “ns” indicates statistical nonsignificant differences. “None” refers to cultures with no magnetic exposure. “Up” and “Down” refer to cultures that were exposed to 1.5 mT PEMFs in the upward and downward direction, respectively.

FIG. 5 shows that TRPC channel and myogenic protein expression is more strongly enhanced by pCM delivery. Relative protein expression (normalized to GAPDH) for A) TRPC1, B) TRPC3, C) TRPC6, D) TRPM7, E) Cyclin B1, F) Cyclin D1, G) p21, H) MyoD and I) HTRA1 from cells administered conditioned media harvested from myoblasts exposed to downward (pCM; solid light grey) or upward (pCM; vertical light grey stripes) magnetic fields expressed as fold change relative to cells given cCM (0 mT; solid dark grey). Data represents the average of n=5-6 biological replicates. Protein expression was determined 24 h after the provision of the respective conditioned media. All magnetic exposures consisted of PEMFs at an amplitude of 1.5 mT applied once for 10 min. Error bars represent the standard error of the mean, with *p<0.05, ** p<0.01, and *** p<0.001 or as indicated, analyzed using multiple paired t-tests. “ns” indicates statistical nonsignificant differences. “None” refers to cultures given cCM. “Up” and “Down” refer to cultures given pCM from donor cells that were exposed to 1.5 mT PEMFs in the upward and downward direction, respectively.

FIG. 6 shows that direct magnetic exposure creates oxidative stress, whereas secretome delivery does not, and is modulated by cell density. A) ROS production in response to direct PEMF exposure. Myoblasts were seeded at a cell density of 3,000 or 6,000 cells/well from stock cultures grown for 2 days B) or 3 days C). Bar charts show the relative fold change of DCF fluorescence intensity normalized to unexposed 3,000 cells/well condition (dark grey bar, left). D) ROS production in response to the administration of cCM or pCM delivered to naïve myoblasts in 96-well plates was measured and expressed as the relative DCF fluorescence intensity normalized to cCM E), as indicated. ROS measurements were taken 4 h post PEMF exposure. F) pCM proliferative capacity is attenuated by high cell density at the time of media collection. Bar chart showing the pooled data for recipient cell numbers expressed as fold change relative to their respective recipient cells (control) in unexposed cCM at 1× or 5× cell densities. Data represents the average of n=3 biological replicates. Error bars represent the standard error of the mean, with *p<0.05, ** p<0.01, *** p<0.001 and ^{#}p<0.0001, analyzed using One-Way ANOVA with Sidak's multiple comparison tests. “ns” indicates statistical nonsignificant differences. “None” refers to cultures given cCM or no magnetic exposure. “Up” and “Down” refer to cultures given pCM from donor cells that were exposed to 1.5 mT PEMFs in the upward and downward direction, respectively.

FIG. 7 shows that pCM collected from magnetically-stimulated myoblasts and myotubes promote myogenic differentiation and survival. A) (i) Myogenic differentiation in response to myoblast pCM. DMEM was conditioned from exposed/unexposed myoblasts in suspension and then supplemented with 2% HS before administering to myoblast after 4 days in culture. (ii) Confocal images and H&E staining of myotubes 4 days post-pCM administration showing nuclear localization of myogenin and myotube densities. (iii) Ratio of myogenin positive nuclei normalized to cytoplasmic intensity (n=>50 cells/condition). Scale bar=120 μm. (iv) Myotube fusion index. Scale bar=100 μm. Relative protein expression of Desmin (v) and Cyclin D (vi) (n=3 biological replicates). B) (i) Myogenic differentiation in response to pCM from myotubes. (ii) Myoblast proliferation in response to myotube pCM. Protein expression of the proliferation markers, Cyclin B1 (iii), Cyclin D1 (iv), p21 (v), and differentiation markers, MyoD (vi), MyoG (vii) and Desmin (viii), analyzed 24 h post-pCM incubation (n=6 biological replicates/condition). C) Myoblast survival in response to pCM with and without FBS supplementation. (i) Myoblast proliferation in response to basal media (DMEM without FBS), growth media (DMEM plus 5% FBS) or pCM from myoblast in serum-free DMEM (n=4 biological sets with 3 technical replicates/condition). Relative abundance of phosphorylated ERK (ii) and JNK (iii) normalized to total ERK or JNK proteins, respectively (n=3 biological replicates per condition). Error bars represent the standard error of the mean, with *p<0.05, ** p<0.01, *** p<0.001 and ^{#}p<0.0001, analyzed using One-Way ANOVA with Sidak's multiple comparison tests. “ns” indicates statistical nonsignificant differences. “None” refers to cultures given cCM. “Up” and “Down” refer to cultures given pCM from donor cells that were exposed to 1.5 mT PEMFs in the upward and downward direction, respectively.

FIG. 8 shows porcine myoblast responses to directional magnetic fields and pCM. Different experimental paradigms on porcine myoblasts (schematics). A) Porcine myoblast proliferation in response to no exposure, or direct down, up, or horizontal exposure, as indicated. Porcine myoblast (recipient) proliferation in response to C2C12 donor pCM B) or porcine donor pCM C) was generated under the different field orientations, as indicated. D) Porcine myoblast (recipient) proliferation in response to EVs isolated from C2C12 donor pCM. Cell enumeration was conducted 24 h post magnetic stimulation A) or 24 h post pCM or EV administration (B, C and D). All data represent the average of n=3 to 4 biological replicates with each consisting of 3 technical replicates. Error bars represent the standard error of the mean, with *p<0.05, *** p<0.001 and ^{#}p<0.0001, analyzed using One-Way ANOVA with Sidak's multiple comparison tests. “ns” indicates statistical nonsignificant differences. “None” refers to cultures given cCM or no magnetic exposure. “Up,” “Down” and “Hori” refer to cultures given pCM from donor cells that were exposed to 1.5 mT PEMFs in the upward, downward and horizontal direction, respectively.

FIG. 9 depicts secretome characterization. (A) Schematic depiction of the experimental paradigm used for the generation of EV and EV-depleted pCM to assess the proliferation responses of C2C12 (recipient) myoblasts. C2C12 myoblasts in suspension were exposed to PEMFs and then allowed to condition the media for 1 h (37° C. in the incubator) prior to EV isolation. (B) Myoblast proliferation assessment 24 h following EV or EV-depleted pCM provision to recipient myoblasts (n=4 biological replicates with each consisting of 3 technical replicates). (C) Transmission electron microscopy (TEM) images of EVs isolated from C2C12 cCM and pCM. Scale bar=100 μm. (D) Flow cytometric analysis of C2C12 EVs using fluorescent-labeled CD9 and CD81. Detection levels given per 500,000 events. (E) Representative western blot images of EV marker, CD9 and cytoskeletal and endoplasmic reticulum markers, actin and calnexin, demonstrating the absence of cellular contamination of the EV preparation. (F) Representative NTA histogram showing size distribution and abundance of C2C12 EVs. (G) Quantitative analysis of cCM and pCM soluble factors using multiplex immunoassay analysis (n=9 biological replicates each consisting of 3 technical replicates). Error bars represent the standard error of the mean, with *p<0.05, ** p<0.01, *** p<0.001, analyzed using One-Way ANOVA with Sidak's multiple comparison tests. “ns” indicates statistical nonsignificant differences. “None” refers to cultures given cCM or no magnetic exposure. “Up” and “Down” refer to cultures given pCM from donor cells that were exposed to 1.5 mT PEMFs in the upward and downward direction, respectively.

FIG. 10 shows that cell orientation influences but is not an absolute determinant of efficacy to magnetic field directionality. (A) Schematic depiction of induced current path in response to the direction of magnetic field stimulation. Unstimulated cells show no induced current path (i). Vertical cells stimulated with either up (ii) or down fields (iii) in the same direction of cell orientation exhibit “short” induced current paths (light grey arrows). (B) Schematic depiction of vertically standing flasks under basal conditions (dark grey) or upon magnetic stimulation (light grey) in the upward (vertical stripes) or downward (solid) field directions. During magnetic field exposure, the flask was completely filled with culture media to keep the cells from drying out; following exposure excess culture media was then removed to allow the cells to grow horizontally for 24 h before growth assessment. (C) Quantification of induced proliferation for cultures exposed to upward (vertical light grey stripes) or downward (solid light grey) PEMFs while in standing flasks expressed as fold change relative to the unexposed scenario (solid dark grey). (D) Schematic depiction of “long” induced current path in response to magnetic field stimulation perpendicular to cell axis. Unstimulated cells show no induced current path (i) and (iii). Regardless of cell axis, cells will respond with a long induced current when exposed to magnetic fields perpendicular to cell axis (iii) and (iv). (E) Experimental paradigm used to test whether the field-flask cross alignment is independent of field directionality. Flasks were either placed in the typical horizontal position or rotated 90 degrees into a standing position and received downward (solid light grey) or horizontal (horizontal light grey line) magnetic field exposure, respectively, maintaining field-flask cross alignment, but changing field directionality. (F) Quantification of induced proliferation for the downward (solid light grey) or horizontal (horizontal light grey stripes) exposure scenarios expressed as fold change relative to their respective unexposed scenarios (solid dark grey). Downward field stimulation produced a significantly greater growth enhancement than horizontal field stimulation, despite maintaining field-flask cross alignment. All data represent the average of n=10 biological replicates, with each biological replicate derived from the means of three technical replicates. All magnetic exposures consisted of PEMFs at an amplitude of 1.5 mT applied once for 10 min. Error bars represent the standard error of the mean, with ** p<0.01, *** p<0.001, and ^{#}p<0.0001 analyzed using One-Way ANOVA with Sidak's multiple comparisons test. “ns” indicates statistical nonsignificant differences. “None” refers to cultures given cCM or no magnetic exposure. “Up,” “Down” and “Hori” refer to cultures given pCM from donor cells that were exposed to 1.5 mT PEMF in the upward, downward and horizontal direction, respectively.

FIG. 11 shows that proliferation enhancement preferentially conferred by downward-directed PEMF exposure occurred independently of the device used. Photographs of in vitro coil system (A), animal coil system (B) and human leg coil system (C). D) Quantification of induced proliferation for cultures that were exposed to downward (solid light grey) or upward (vertical light grey stripes) PEMFs for the 3 different magnetic field devices expressed as fold change relative to their respective unexposed scenario (0 mT). Data represents the average of n=2-10 biological replicates, with each biological replicate derived from the means of three technical replicates. A u metal case that typically houses the in vitro coil system (A) was removed to display the coil arrangement. E) Photograph of human arm coil system and associated quantification (F) of induced proliferation for cultures that were exposed to downward (solid light grey), upward (vertical light grey stripes) or horizontal (horizontal light grey stripes) PEMFs expressed as fold change relative to their respective unexposed scenario (0 mT). Data represents the average of n=3 biological replicates, with each biological replicate derived from the means of three technical replicates. Shown within each coil system is a stack of standard T75 culture flasks (˜16×8×4 cm each). The leg (C) and arm (D) coils were positioned vertically for the assessment of up and down field effects; shown here in the horizontal position used for human subjects. G) Photograph of human breast cancer coil system (Tai, Y. K. et al., Front Oncol 11, 783803 (2021)) and associated quantification (H) of induced proliferation for cultures that were exposed to downward (solid light grey) or upward (vertical light grey stripes) PEMFs expressed as fold change relative to their respective unexposed scenario (0 mT). Data represents the average of n=6 technical replicates, derived from two biological replicates. Shown within the breast cancer coil is a stack of standard T25 culture flasks (˜10×4×2.5 cm). In all cases, myoblast cultures were situated within a region of the coil system of field uniformity to assure even exposure of the entire dish (Crocetti, S. et al., PLOS One 8, e72944 (2013)). Error bars represent the standard error of the mean, with #p<0.0001 analyzed using One-Way ANOVA with Sidak's multiple comparisons test. All data shown were generated in response to direct exposure of myoblasts to directionally specified PEMFs at an amplitude of 1.5 mT applied once for 10 min. The Student's paired t-test was used to analyze the mean between downwards and upwards fields for each PEMF device, with *p<0.05. “ns” indicates statistical nonsignificant differences. “None” refers to magnetic exposure. “Up,” “Down” and “Hori,” refer to cultures that were exposed to 1.5 mT PEMFs in the upward, downward and horizontal direction, respectively.

FIG. 12 depicts pCM myokine expression. Heat map showing average fold change of analytes from conditioned media generated from myoblasts in suspension in response to downward, upward and no magnetic exposure. Fold change for each analyte is derived by taking the mean fold change over 0 mT unexposed condition (Wong C. J. K. et al., Biomaterials 6; 287:121658 (2022)).

FIG. 13 depicts the kinetics of PEMF-induced proliferation. C2C12 myoblasts were magnetically stimulated 24 h post seeding and kinetically tracked over 3 days using Cytosmart Lux2. Data presented are from two independent Cytosmart devices, tracking the simultaneous proliferation of C2C12 myoblasts after 0 mT or 1.5 mT downward fields exposure. The cell coverage (%) data is compiled from the algorithm developed by Cytosmart, and shows accelerated proliferation in myoblasts exposed to 1.5 mT down fields compared to unexposed 0 mT.

FIG. 14 depicts the efficacy window in upwards and downwards direction for myoblasts in suspension. When exposed in the downward direction, the peak response was at 1.5 mT. When exposed in the upward direction, there was no evident peak up to 3 mT. However, the response at 3 mT was still lower compared to exposure at 1.5 mT in the downward direction. Myoblast proliferation assessment was carried out 24 h following PEMF exposure to myoblasts (n=6 biological replicates with each consisting of 3 technical replicates). Error bars represent the standard error of the mean, with *** p<0.001, **** p<0.0001, analyzed using One-Way ANOVA with Sidak's multiple comparisons test. “ns” indicates statistical nonsignificant differences. “None” refers to cultures given no magnetic exposure. “Up,” “Down” refer to cultures that were exposed to 1.5 mT PEMF in the upward and downward, respectively.

FIG. 15 depicts a bar graph of myoblast survival in response to EV fraction of pCM with and without FBS supplementation. Myoblast proliferation (live cell count) in response to basal media (DMEM without FBS), growth media (DMEM plus 10% FBS) or EVs from myoblast in serum-free DMEM (n=3 biological replicates with each consisting of 3 technical replicates). Error bars represent the standard error of the mean, with ** p<0.01, *** p<0.001, analyzed using One-Way ANOVA with Sidak's multiple comparisons test. “ns” indicates statistical nonsignificant differences. “None” refers to cultures given exosomes isolated from pCM with no magnetic exposure. “Up,” “Down” refer to cultures that were given exosomes isolated from pCM that was exposed to 1.5 mT PEMF in the upward and downward, respectively.

FIG. 16 is a bar graph depicting the storage and time of conditioning for optimal EV potency. Exosomes (EVs) stored at 4° C. lose their potency within a week. Constitutive release of exosomes (hatched dark grey; 0 mT) decreases after 20-30 min of conditioning, whereas magnetically induced (hatched light grey; 1.5 mT) exosome release increased and was maximal at 60 min. Myoblast proliferation assessment (live cell count) was carried out 24 h following the addition of fresh or one week-old exosomes isolated from exposed myoblasts to naïve recipient myoblasts (n=3 biological replicates with each consisting of 3 technical replicates). “None” refers to cultures given exosomes isolated from pCM and no magnetic exposure. “Down” refers to cultures that were given exosomes isolated from pCM and then exposed to 1.5 mT PEMF in the downward direction.

FIG. 17 shows that optimal time of conditioning for isolation of exosomes to induce proliferation in naïve recipient myoblasts was for 60 min. C2C12 myoblasts were exposed to PEMF and incubated for 30 min to 120 min prior to the start of exosome isolation. Myoblast proliferation (live cell count) assessment was carried out 24 h following addition of exosomes isolated from exposed myoblasts to naïve recipient myoblasts (n=3 biological replicates with each consisting of 3 technical replicates). Error bars represent the standard error of the mean, with and data was analyzed using One-Way ANOVA with Sidak's multiple comparisons test.

FIG. 18 shows a method to increase the anti-cancer potency of the muscle secretome. A) Breast cancer (67NR mouse cell line) survival after administration of conditioned media (CM) harvested from myotubes. Myotubes were preconditioned, or not, with myoblast secretome as indicated. Myotube Secretome for Anti-cancer Effect: Preconditioning Scenario. cCM: CM from unexposed cells; pCMu: CM from cells exposed to 1.5 mT upward PEMFs: pCMd: CM from cells exposed to 1.5 mT downward PEMFs; 0: no secretome provisioned. Secretome was harvested from unexposed (0 mT; dark grey) or 1.5 mT PEMF-exposed (downward; light grey) myotubes and then administered to breast cancer cells. B) Table showing the effect of preconditioned muscle secretome on survival of breast cancer cells. Media control=“0” in A. All cell numbers were normalised (fold change) to that observed in response to naïve (unexposed) myotube conditioned media without pre-conditioning with myoblast secretome (cCM: 0). Data represents the average of n=5 biological replicates, where **** p<0.0001 analysed using One-Way ANOVA with Sidak's multiple comparisons test.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### Definitions

For convenience, certain terms employed in the specification, examples and appended claims are collected here.

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, “a” or “an” may mean one or more than one unless indicated to the contrary or otherwise evident from the context. The inventors found that the more enzyme used the faster the reaction proceeded.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “promoting proliferation”, “enhancing proliferation” or any other of equivalent grammatical meaning thereof may be used interchangeably and refer to the improvement of one or more characteristics and/or functions of cellular proliferation (i.e., the process of generating an increased number of cells through cell division) in a treated/recipient cell as compared to a control cell (for example, a cell cultured in the conditioned medium of the present invention compared to a control cell cultured in normal growth medium). Examples of characteristics and/or functions of cell proliferation would be understood by those skilled in the art to include, but not limited to, rate of cell division, rate of cell growth, cell size, upregulation of certain proliferative signalling, downregulation of growth suppressors etc.

As used herein, the term “promoting differentiation”, “enhancing differentiation” or any other of equivalent grammatical meaning thereof may be used interchangeably and refer to the improvement of one or more characteristics and/or functions of cellular differentiation (i.e., the process of converting one cell type into another cell type, typically from an immature unspecialized, cell to a mature, specialized form and function) in a treated/recipient cell as compared to a control cell (for example, a cell cultured in the conditioned medium of the present invention compared to a control cell cultured in normal growth medium). Examples of characteristics and/or functions of cell differentiation would be understood by those skilled in the art to include, but not limited to, rate of change, change in morphological structures such as cell shape, cell size, membrane potential, and metabolic activities, upregulation of certain differentiation-related signalling etc.

As used herein, the term “promoting senescence”, “enhancing senescence” or any other of equivalent grammatical meaning thereof may be used interchangeably and refer to the improvement of one or more characteristics and/or functions of cellular senescence (i.e., the process by which a cell ages and permanently stops dividing but does not die) in a treated/recipient cell as compared to a control cell (for example, a cell cultured in the conditioned medium of the present invention compared to a control cell cultured in normal growth medium). Examples of characteristics and/or functions of cell senescence would be understood by those skilled in the art to include, but not limited to, morphological changes such as flattened and enlarged morphology, presence of molecular markers such as senescence-associated heterochromatin foci (SAHF), expression of tumour suppressors and cell cycle inhibitors etc.

A description of exemplary, non-limiting embodiments of the invention follows.

The present invention is based, in part, on the discovery that magnetic field directionality affects secretome response in a manner determined by accepted mitohormetic principles. In particular, the inventors have found that the application of pulsing electromagnetic fields (PEMFs) of different directionalities induce different levels of reactive oxygen species (ROS)/mitochondrial respiration from secretome donating cells. As such, changes in field directionality can advantageously be used to produce secretomes of different characteristics from the same secretome donating cells. In this regard, the inventors have successfully employed a brief and non-invasive PEMF-exposure paradigm and developed, inter alia, a conditioned media capable of inducing secretome production and release in recipient cell cultures.

To this end, provided in one aspect of the present disclosure is a method of producing a conditioned medium capable of promoting proliferation, differentiation or senescence of progenitor and/or stem cells, wherein the method comprises the steps of:
a) culturing proliferating, differentiating or senescent (oxidatively stressed) progenitor and/or stem cells in media as a suspension culture in a bioreactor;
b) exposing the proliferating, differentiating, or senescent (oxidatively stressed) progenitor and/or stem cells to downward-directed or upward-directed low amplitude pulsed electromagnetic fields (PEMFs); and
c) collecting the PEMF-conditioned media (pCM) which comprises a secretome that reflects the status of the cells in the bioreactor and has proliferation-promoting capability, cell survival-promoting capability, cell differentiation-promoting capability, or cell senescence-promoting capability.

Advantageously, the provision of the pCM produced by the methods of the present invention on recipient cells does not trigger oxidative stress, as opposed to the application of direct magnetic exposure which may produce mild oxidative stress, thereby providing a survival advantage on recipient cells.

The methods described herein may be adapted to produce a pCM comprising a secretome of a specific characteristic for a specific developmental objective (such as proliferation, differentiation or senescence), by modulating the direction of magnetic field exposure used.

For example, the application of a downward magnetic field may produce a pCM capable of enhancing proliferation. In another example, the application of a downward magnetic field may also produce a pCM capable of enhancing differentiation. In another example, the magnetic field direction may be switched from up to down to obtain a pCM with senescence or proliferative capabilities.

It would be appreciated that the secretome of a cell is state-specific (i.e., the cell secretome mirrors the status of the cell). Cells in proliferative state may produce a secretome that promotes proliferation, while cells undergoing differentiation may produce a secretome that inhibits proliferation and enhances differentiation. Accordingly, the methods disclosed herein may also be advantageously adapted to provide a pCM comprising state-specific secretome, such as by employing secretome-donating cells of a particular cellular state. For example, the methods herein may employ differentiated myotubes to obtain pCM capable of enhancing differentiation in proliferating myotube cells. Secretome donating cells may also first be cultured and grown to a specific cell status (e.g., a proliferative state, a differentiated state, or a senescent state, etc.) prior to magnetic field exposure in order to obtain a secretome of the same state. In this regard, pCM comprising secretome collected from proliferating donor cells may preferentially promote proliferation, pCM comprising secretome collected from differentiating donor cells may preferentially promote differentiation and the pCM comprising secretome collected from senescent donor cells may preferentially promote senescence.

In some embodiments, the pCM from proliferating progenitor and/or stem cells exposed to downward-directed or upward-directed low amplitude PEMFs may promote proliferation, with the exception that pCM from proliferating myoblasts exposed to downward-directed PEMFs will promote differentiation. In other embodiments, the pCM from differentiating progenitor and/or stem cells exposed to downward-directed low amplitude PEMFs may promote differentiation, whereas exposure to upward-directed PEMFs will promote proliferation and/or survival. In some other embodiments, the pCM from senescent (oxidatively stressed) progenitor and/or stem cells exposed to downward-directed or upward-directed low amplitude PEMFs to promote senescence. In this regard however, upward-directed low amplitude PEMFs are less efficient than downward-directed low amplitude PEMFs at activating mitochondrial oxygen-based respiration and generating reactive oxygen species (ROS) and thus may require administration at a higher amplitude than downward-directed low amplitude PEMFs to have a similar effect.

A person skilled in the art would appreciate that in accordance with the mitohormetic principles, a mild PEMF exposure may induce low levels of oxidative stress that are adaptive and that stimulate the cell secretome, while stronger PEMF exposure may produce greater levels of oxidative stress that are instead damaging and detrimental to the cell's survival. Accordingly, the parameters of PEMF exposure may be modulated to optimise secretome production and release. In some embodiments, the progenitor and/or stem cells may be exposed to the PEMFs for a single 10-30-minute duration, a single 10-25-minute duration, a single 10-20-minute duration, a single 10-15-minute duration or a single 10-minute duration. In particular, the progenitor and/or stem cells may be exposed to the PEMFs for a single 10-minute duration. Preferably, the donor progenitor and/or stem cells may be exposed to the PEMFs for no less than 10 min and/or no longer than 30 min. In this regard, it would be appreciated that a shorter duration of magnetic exposure may be insufficient to condition the medium while a longer duration of exposure may result in stress factors being released and thus contaminate said medium. In some embodiments, a minimum of at least 10 min of PEMF exposure may be required to obtain the most efficacious secretome production and release.

Apart from the direction and duration of the PEMF exposure, the power and pulsing rate of the PEMF may also be modulated. The PEMFs to be applied may be at an amplitude of 0.5-4.0 mT, 0.5-3.5 mT, 0.5-3.0 mT, 0.5-2.5 mT, 0.5-2.0 mT, 0.5-1.5 mT, 0.5-1.0 mT, 1.0-3.5 mT, 1.5-3.5 mT, 2.0-3.5 mT, 2.5-3.5 mT, 3.0-3.5 mT, 1.0-2.5 mT, 1.5-2.5 mT or 2.0-2.5 mT. Similarly, the PEMFs may be applied in 20×150 μs on and off pulses for 6 ms at a repetition frequency of 15 to 50 Hz.

In some embodiments, the progenitor and/or stem cells may be exposed to the PEMFs (a) for a single 10-30-minute duration, and/or (b) at a downward-directed amplitude of 0.5-2 mT for muscle cells, 0.5-2 mT for fibroblast cells, 0.5-3 mT for hematopoietic stem cells, 2.5-3.5 mT for mesenchymal cells, or 1.5-2.5 mT for dental pulp cells, and/or (c) in 20×150 μs on and off pulses for 6 ms at a repetition frequency of 15 to 50 Hz.

In some embodiments, the methods disclosed herein may produce secretome from cells grown in liquid suspension. Advantageously, cells in suspension are not limited by the caveats imposed by high cell density characteristic of growth on planar surfaces. In contrast, cells attached onto a 2D surface (such as tissue culture plastic) are limited in the density they can achieve, whereas cells in suspension are considered multi-layered, effectively filling more of the liquid space. Cells in suspension may also be collected from cultures in their healthiest state, from cells grown in low to medium density donor cultures. Further, numerous low-density cultures (low confluence with a minimal contact inhibition) may also be harvested and added together to achieve high-density suspension (cell-cell contactless) cultures. In addition, the suspension paradigm also allows for the rapid concentrating of the secreted factors, particularly of extracellular vesicles.

Advantageously, the suspension paradigm disclosed herein may also improve the outcome of proteomic characterisation and analyses as the cells do not have time to respond (in a paracrine manner) adversely to the absence of serum. In addition, the suspension paradigm disclosed herein does not condition the media with de novo stress signalling molecules/metabolites as the duration of conditioning is insufficient for the production of new proteins. Further, suspension cultures are also a cleaner method to separate cells from supernatant (by centrifugation).

In some embodiments, the methods disclosed herein may also produce secretome from progenitor and/or stem cells that are in and/or on free-floating micro scaffolds for liquid suspension cultures. Cells grown on/in micro-scaffolds remain freely floating and can be concentrated in the suspension paradigm, but are attached. More advantageously, cells grown in micro-scaffolds will not experience the stress of enzymatic (e.g., trypsin) detachment from tissue cultures dishes/plates in preparation for resuspension. In various embodiments, differentiated tissues (for example, differentiated myotubes) may also be grown in/on micro-scaffolds.

In some embodiments, the progenitor and/or stem cells may be myoblast cells, neuronal stem cells, hematopoietic stem cells, dental pulp stem cells, fibroblast cells or mesenchymal stromal cells. In some embodiments, the hematopoietic stem cells may give rise to red blood cells, reticulocytes, and/or platelets. In this regard, the red blood cells, reticulocytes, and/or platelets may also be suitable to function as donor cells and be subjected to the PEMF induction paradigm as disclosed in the present invention for the production of a conditioned medium.

In some embodiments, recipient cells may be myoblast cells, neuronal stem cells, hematopoietic stem cells, dental pulp stem cells, mesenchymal stromal cells or fibroblast cells. In some embodiments, the recipient cells may also be red blood cells and other red blood cell types such as reticulocytes and/or platelets.

In some embodiments, the progenitor and/or stem cells may be differentiated, proliferating or senescent cells. Preferably, the progenitor and/or stem cells are differentiated or proliferating.

In some embodiments, the progenitor and/or stem cells in step a) have been prior expanded and/or conditioned to be in a proliferating, differentiating or senescent state in growth media or media of defined composition.

In accordance with the paracrine nature of tissue development, progenitor cells of a particular lineage produce secretomes that are specific and preferential for that cell type without cross-modulation from other tissue types. Advantageously, the pCM obtained by the methods disclosed herein are more effective at improving cellular survival compared to conventional fetal bovine serum. Therefore, the methods of the present invention may be capable of being self-sustainable. Further, with the employment of magnetic field induction of secretome, the methods disclosed herein may also be capable of facilitating and enhancing cell growth and development without the need for supplementation (i.e., without exogenous and expensive growth factors). In some embodiments, the culturing in step a) is in serum-free and exogenous growth and/or trophic factor-free media.

It would be appreciated by a person skilled in the art that the TRPC1 calcium-permeable channel plays an important role in the magnetic mitohormetic responses. TRPC1 channels have been shown to mediate cellular response to PEMF exposure and as such, the presence of TRPC1 channel inhibitor may negatively affect the cell's mitohormetic response when exposed to PEMF. For example, the presence of streptomycin, a TRPC1 channel inhibitor, may prevent PEMF-induced secretome enhancement by blocking calcium entry via TRPC1 channels. Accordingly in some embodiments, the culturing in step a) is performed in the absence of TRPC1 receptor inhibitors, such as aminoglycoside antibiotics. Examples of aminoglycoside antibiotics include, but not limited to, gentamicin, amikacin, kanamycin, tobramycin, neomycin, netilmicin, and streptomycin. Preferably, the aminoglycoside antibiotic is streptomycin.

In another aspect, there is provided a method of proliferating and differentiating progenitor and/or stem cells, comprising adding the pCM from proliferating and differentiating cells, respectively, as defined herein, to a progenitor cell culture.

In some embodiments, the methods of the present disclosure may be adapted for use in cell-based meat applications. In this regard, one of the limiting factors in providing affordable cell-based meat production is the provision of muscle-specific trophic factors. Advantageously, the methods disclosed herein therefore may be adapted to provide both proliferation and differentiation promoting pCMs which would be optimal for cell-based meat applications. Accordingly in various embodiments, the pCM may be produced from myoblasts and may be used to feed a cell-based meat culture.

In another aspect, there is provided a system comprising:
i) a bioreactor within which a first culture of progenitor and/or stem cells in suspension is subjected to downward-directed low amplitude pulsed electromagnetic fields (PEMFs), or upward-directed low amplitude pulsed electromagnetic fields (PEMFs), to produce a PEMF-conditioned media (pCM), and
ii) a second cell culture to be expanded (via induced proliferation) and/or differentiated and/or induced into a senescent (oxidatively stressed) state,
   wherein said PEMF-conditioned media (pCM) from (i) is provided to cell culture (ii).

In some embodiments, the progenitor and/or stem cells are exposed to the PEMFs: (a) for a single 10 to 30-minute duration, and/or (b) at a downward-directed amplitude in the range of 0.5-2 mT for muscle cells, in the range of 0.5-2 mT for fibroblast cells, in the range of 0.5-3 mT for hematopoietic stem cells, in the range of 2.5-3.5 mT for mesenchymal cells, or in the range of 1.5-2.5 mT for dental pulp cells, and/or (c) in 20×150 μs on and off pulses for 6 ms at a repetition frequency of 15 to 50 Hz.

In various embodiments, the progenitor and/or stem cells are in and/or on free-floating micro scaffolds; and/or the progenitor and/or stem cells in step i) have been prior expanded and/or conditioned to be in a proliferating, differentiating or senescent (oxidatively stressed) state in a growth media or media of defined composition; and/or the progenitor and/or stem cells are myoblast cells, neuronal stem cells, red blood cells, dental pulp stem cells, or mesenchymal stromal cells.

In some embodiments, the progenitor and/or stem cells are differentiated or proliferating. In some embodiments, the progenitor and/or stem cells in i) are myoblast cells and the cell culture ii) is for cell-based meat production. In other embodiments, the cells in i) are differentiated myotubes.

In various other embodiments, the culturing in step i) is in serum-free and exogenous growth and/or trophic factor-free media.

In another aspect, there is provided a method of enhancing cultured meat production, comprising feeding a cell-based meat culture a pCM produced by the methods of the invention as disclosed herein.

In some embodiments, the cell-based meat culture is fed: a) a proliferating myoblast-derived pCM to promote proliferation of the cells in said culture, and then b) a differentiating myoblast-derived, or differentiated myotube-derived, pCM to promote differentiation of the proliferating cells in said culture to form a cell-based meat. In other embodiments, the pCM is produced in a serum-free and exogenous growth and/or trophic factor-free media.

In a further aspect, there is provided a PEMF-conditioned media (pCM), produced by the methods disclosed herein, preferably comprising exosomes.

In another aspect, there is provided a method of pre-conditioning proliferating, differentiating or senescent (oxidatively stressed) progenitor and/or stem cells for use in the production of a PEMF-conditioned media (pCM), wherein the method comprises: contacting a sample of proliferating, differentiating or senescent (oxidatively stressed) progenitor and/or stem cells with the pCM as provided in the present invention. Advantageously, exposing donor cells to pCM in their early growth enhances their secretory response upon later exposures to PEMF. In this regard, the cell pre-conditioning paradigm of the present invention thus may provide the production of super secretors.

The methods described herein are robust and time-saving, requiring only a short time period to condition the medium to release most of the cells' secretome allotment. Rapid collection of the media also avoids the possibility of the cells depleting the media of generated factors they secreted by consuming the same factors themselves. In this regard, the immediate release of nearly the complete allotment of the secretome in response to magnetic exposure assures stage-specific properties, a snapshot of the cell's agenda at the time of exposure. In contrast, delaying collection of the secretome (to allow accumulation of the secretome components to appreciable levels) may allow for the biosynthesis of new secretome agents to occur and will, in essence, contaminate the secretome pool; providing mixed messages.

The methods described herein also advantageously provide secretome specificity and autologous secretome production. Secretome can be collected from defined cells of interest or at a defined state of development, thereby eliminating secretome contamination from other cell types and from cells in heterogeneous states of development. Further, collecting secretome from cells in conventional tissue culture is a more protracted process and risks the cells undergoing heterogeneous levels of development.

Progenitor cells that are stressed, underfed, or overgrown produce an inflammatory secretome that confers senescence are known as the Senescence-Associated Secretory Phenotype (SASP). As such, the methods described herein may also be adapted to avoid senescence and provide a pCM comprising growth promoting secretome. In this regard, secretome donating cells can be harvested from cultures that are at low density (healthiest state) and concentrated in suspension for subsequent secretome collection.

On the other hand, the methods disclosed herein may also be suitably adapted to obtain senescent-state secretome for use in anti-cancer applications. For example, the provision of pCM comprising the SASP secretome may be used to stall cancer growth.

As disclosed herein, the methods of the present invention may be suitable for use in broad applications, such as in regenerative medicine, cosmetic, as well as in the cell-based meat industries.

For example, stem cell secretome collection is commonly used in the cosmetic industry. Thus, the methods described herein may be adapted to supply the secretome of interest at an industrial level.

Similarly, the methods of the present invention may also be adapted for use in the regenerative medicine, such as in the provision of pCM comprising healing factors to stimulate regeneration in injured tissues. For example, the methods disclosed herein may be used to produce magnetically-induced platelet rich plasma (PRP) secretome, which is widely used for its high efficacy in promoting wound healing. In another example, the methods disclosed herein may also be applied to produce magnetically-induced dental pulp stem cell secretome for the treatment of injured motor neurons. In a further example, the methods disclosed herein may also be suitably adapted for anti-cancer applications. In this regard, a field directionality that produces excessive oxidative stress may cause the induction of cell senescence. Accordingly, the pCM comprising the SASP secretome may be used to stall cancer growth.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1: Materials and Methods

### C2C12 Cell Culture and Chemical Reagents

C2C12 mouse skeletal myoblasts were obtained from American Type Culture Collection (ATCC; LGC Standards, Teddington, United Kingdom) and cultured in Dulbecco's Modified Eagle Medium (DMEM) (Thermo Fisher Scientific, Waltham, MA, USA) containing 10% fetal bovine serum (FBS) (Biowest, Nuaille, France), and maintained in a humidified incubator with 5% CO₂. Cells were passaged every 48 h to maintain them below 40% confluence, unless otherwise explicitly stated. Myoblasts were seeded into tissue culture dishes/flasks at 3,000 cells/cm² (Yap, J. L. Y. et al., Faseb j 33, 12853-12872 (2019)), unless otherwise explicitly stated. Twenty-four hours later the plates were exposed to pulsed electromagnetic fields (PEMFs). Cell number in each well of a 6-well plate was determined using enzymatic dissociation with TrypLE Express Enzyme (Thermo Fisher Scientific, Waltham, MA, USA), followed by the standard trypan blue exclusion method, and counted with a hemocytometer.

### Porcine Myoblast Culture and Chemical Reagents

Passage 17 (P17) porcine myoblasts were grown in growth media consisting of DMEM high glucose (Gibco #1280017), 15% FBS (Hyclone #SV30160.03HI), 1% penicillin-streptomycin (Sigma, #P4333) and 10 ng/ml Fibroblast Growth Factor 2 (FGF2) (Invitrogen, Carlsbad, CA, USA, PHG0360). Upon reaching 80% confluency in a 10 cm plate, porcine myoblasts were trypsinized using 3 ml of TrypLE™ (Thermo Scientific #12605010) and incubated at 37° C. for 5 min. 10 ml of porcine full growth media was added to neutralize the TryPLET and the cell suspension was centrifuged at 1,500 rpm for 5 min to obtain the cell pellet. The cell pellet was then resuspended in 5 ml of fresh porcine growth media without the addition of penicillin-streptomycin. Cell counting was performed using a hemocytometer and a total of 60,000 cells were seeded into each well of 6-well plates containing 2 ml of myoblast full growth media without penicillin-streptomycin. The porcine myoblasts were then left to incubate overnight at 37° C. in a standard tissue culture incubator.

### Pulsed Electromagnetic Fields (PEMFs) Exposure

The PEMF signal used in this study has been described and myogenically characterized in previous studies (Yap, J. L. Y. et al., Faseb j 33, 12853-12872 (2019); Tai, Y. K. et al., Faseb j 34, 11143-11167 (2020), incorporated herein by reference). Briefly, the employed PEMF devices produce spatially homogeneous, time-varying magnetic fields, consisting of barrages of 20×150 μs on and off pulses for 6 ms at a repetition frequency of 15 or 50 Hz. The magnetic flux density plateaued at a predetermined amplitude of 1.5 mT within ˜50 ms (˜17 T/s). As previously described (Crocetti, S. et al., PLOS One 8, e72944 (2013)), all tissue culture flasks, dishes or tubes were placed within a region of greatest magnetic field uniformity within which the entirety of the vessel is exposed evenly to the traversing magnetic field lines. All PEMF-treated samples were compared with time-matched control samples (0 mT) that were manipulated in the same way as the experimental samples, including placement into the PEMF-generating apparatus for the designated time, except that the apparatus was not set to generate a magnetic field. All magnetically-stimulated samples in this study were exposed once for a duration of 10 min to 1.5 mT amplitude PEMFs of different field line directionalities.

### Field Directionality

Employed in this study were an in vitro coil system (Coil 1) (Yap, J. L. Y. et al., Faseb j 33, 12853-12872 (2019)), animal coil system (Coil 2) (Fields at Work, Zürich), human leg coil system (Coil 3) (FLEX LTD. Singapore), human arm coil system (Coil 4) (HOPE Technik PTE. LTD. Singapore) and human breast cancer coil (Coil 5) (Tai, Y. K. et al., Front Oncol 11, 783803 (2021)). All coil systems were designed to generate analogous magnetic fields as described above. PEMF Coil systems 1 and 2 were designed with an in-built capability to electronically switch field directionality, upwards or downwards. Briefly, the directionality of field exposure is a function of the direction of the current flowing through the field generating coil sub-assemblies. By changing the direction of the current flow, the field direction can be inverted. A polarity-switching H-bridge was implemented to allow for the switching of field directionality by reversing the signal current applied to the coil system. Changes in magnetic field directionality with PEMF coil systems 3, 4 and 5 were accomplished manually by changing the orientation of the coil system relative to the culture flask placed within the lumen of the coils. Field uniformity produced by the distinct coil systems was routinely validated using an ExpoM-ELF (Fields at Work, Zurich) low frequency magnetic fields exposure meter. Magnetically-induced C2C12 murine muscle cell proliferation enhancement was similar in magnitude with all PEMF devices.

Myoblast cultures were exposed to one or more of the following conditions: 1) unexposed 0 mT; 2) 1.5 mT exposure in the horizontal direction; 3) 1.5 mT exposure in the downward direction and; 4) 1.5 mT exposure in the upward direction. Culture dishes were placed within the indicated coil system in the intended horizontal (flat) position with the magnetic field lines oriented either parallel or perpendicular to the long axis of the plate, unless otherwise explicitly stated (see next).

### Determination of PEMF Efficacy Window

Myoblast cultures were exposed to one or more of the following PEMF conditions for 10 min: 1) unexposed 0 mT; 2) 0.5 mT in the downward direction; 3) 0.5 mT in the upward direction; 4) 1.5 mT in the downward direction; 5) 1.5 mT in the upward direction; 6) 3 mT exposure in the downward direction and; 7) 3 mT exposure in the upward direction. The cultures were incubated in a 37° C. incubator for 24 h before enumeration using a Trypan Blue assay.

### Interaction Between Flask Orientation and Magnetic Field Directionality

Cells were seeded at 3,000 cells/cm² into T25 flasks and 24 h later exposed to 1.5 mT amplitude PEMFs. The directionality of the PEMF field lines applied to the flasks were either: 1) horizontal; 2) downward or; 3) upward direction. To study the interplay between flask orientation and magnetic field direction, culture T25 flasks were exposed to PEMFs of the above directionalities while either lying in the prescribed horizontal position or in a standing upright (vertical) position. While in the standing orientation, the flask and cells were temporarily filled with media to the brim to prevent the adhered cells from drying out while the flasks were in the upright position. Immediately after PEMF exposure, excess media was removed from the standing flasks, leaving only 5 ml media in each flask to nurture cell growth while in a conventional horizontal position within a tissue culture incubator. Unexposed flasks represented the 0 mT control scenarios and were in either horizontal or standing positions as indicated in the applicable figure legend. Cell number was determined after 24 h of growth.

### PEMF-Conditioned Media and Washout Experiments in Adherent Cultures

Twenty-four hours post-cell seeding, donor cultures grown in flasks were exposed to PEMFs of the indicated direction for 10 min or placed within the unpowered coil system for 10 min to serve as the control condition. CM from the donor cells was collected 6 h post-conditioning and given to age-matched naïve recipient cultures (scenario 1). For CM washout experiments (scenario 2), CM from PEMF-exposed cells were removed 1 h post-PEMF exposure and replaced with age-matched media from unexposed sister cultures. Cell counts were performed on the recipient cultures 24 h following the transfer of CM (scenario 1) or the delivery of age-matched naïve media (scenario 2).

### Cell Suspension Experiments and PEMF-Conditioned Media Collection

Cells in suspension were generated by enzymatically dissociating adherent cells from T75 flasks and resuspending them into 24 ml of growth media (DMEM+10% FBS) at a concentration of 170,000 cells/ml media. The cell suspension was then subdivided into 4 independent 50 ml conical tubes and exposed to either one of these conditions, (1) 0 mT, 2) 1.5 mT horizontal direction, (3) 1.5 mT downwards direction, or (4) 1.5 mT upwards direction. The cells were then plated into a 6-well plate at a density of 3,000 cells/cm² and allowed to grow for 24 h before counting.

Following trypsinization and washing, cells were resuspended in 12 ml of complete DMEM (1× cell density) and immediately exposed to PEMFs of the indicated direction for 10 min. Thereafter, cells were allowed to recover in a standard tissue culture incubator for 30 min and to condition the bathing media. The cell suspension was then centrifuged at 1,200 rpm for 5 min and the supernatant (CM) was given to pre-plated naïve recipient cultures and allowed to grow for 24 h later before counting. For 5× experiments, the number of cells in suspension was 5-fold more than the usual 1× density, reconstituted in the same 1× volume of complete media used as CM to be given to pre-plated naïve cells for cell counting. The final concentration of cells for the 1× and 5× conditions were 15,000/ml and 75,000/ml, respectively.

### Streptomycin Treatments

For experiments with adherent cultures, streptomycin (0.1 mg/ml; Merck, Germany) was added directly to the cell culture media 2 h before PEMF exposure. Six hours following exposure, the conditioned media was harvested from donor cultures (magnetically exposed) and given to recipient cultures in replacement for their existing media and allowed to grow for 24 h before cell enumeration. For cell suspension experiments, streptomycin (0.1 mg/ml) was added at the time of cell resuspension, immediately followed by PEMF exposure. The cells were then allowed to recover in a standard tissue culture incubator for 30 min before centrifugation at 1,200 rpm for 5 min prior to the collection of the supernatant to be used as conditioned media on pre-plated naïve recipient cultures. The recipient cultures were allowed to grow for 24 h before cell counting.

### Western Blot Analysis of Whole Cell Lysates

Protein extraction was performed using RIPA lysis buffer containing 50 mM NaCl, 1 mM EDTA, 50 mM Tris-HCl, 1% Triton X-100, 0.05% SDS, EDTA-free 1× protease inhibitor cocktail (Nacalai Tesque Inc., Japan), 1× PhosSTOP™ phosphatase inhibitor (Merck, Germany) and 0.1% sodium deoxycholate (Merck, Germany). Whole cell lysates were collected using a cell scraper and incubated at 4° C. for 30 min before being spun at 12,000 rpm for 15 min at 4° C. Protein concentration was determined using Pierce BCA Protein Assay Kit (Thermo Fisher Scientific, USA). Whole cell lysates were prepared in 4× Laemmli buffer with added β-mercaptoethanol (Bio-Rad Laboratories and Sigma Life Science, USA, respectively) and were boiled at 95° C. for 5 min. 20-25 μg of proteins from the whole-cell lysates were resolved using denaturing and reducing SDS-PAGE and transferred to PVDF membrane (Thermo Fisher Scientific, USA). The antibodies and dilution factors used are listed in Table 1.

TABLE 1
List of antibodies, vendors and employed dilution factors.

| | | Dilution |
|---|---|---|
| Protein target and catalogue number | Vendor, Country | Factor |
| Cyclin-dependent kinase inhibitor (P21) | Santa Cruz Biotechnology, Texas, | 1:300 |
| (sc-6246) | USA | |
| Cyclin B1 (CB1) (CST 4138) | Cell Signaling Technology, | 1:1,000 |
| | Massachusetts, USA | |
| Cyclin D1 (CD1) (sc-8396) | Santa Cruz Biotechnology, Texas, | 1:300 |
| | USA | |
| HTRA serine peptidase 1 (HTRA1) | Proteintech, Chicago, USA | 1:1,000 |
| (55011-1-AP) | | |
| Transient receptor potential canonical 1 | Santa Cruz Biotechnology, Texas, | 1:300 |
| (TRPC1) (sc-133076) | USA | |
| Transient receptor potential canonical 3 | Santa Cruz Biotechnology, Texas, | 1:300 |
| (TRPC3) (sc-514670) | USA | |
| Transient receptor potential canonical 6 | Santa Cruz Biotechnology, Texas, | 1:300 |
| (TRPC6) (sc-515837) | USA | |
| Transient receptor potential cation channel | Alomone, Israel | 1:200 |
| subfamily M member 7 (TRPM7) (ACC- | | |
| 047) | | |
| Myoblast determination protein 1 (MyoD) | Santa Cruz Biotechnology, Texas, | 1:300 |
| (sc-71626) | USA | |
| Myogenin (MyoG) (immunofluorescence | Santa Cruz Biotechnology, Texas, | 1:20 (IF), |
| and western blotting) (sc-12732) | USA | 1:300 (WB) |
| Glyceraldehyde-3-phosphate | Proteintech, Chicago, USA | 1:10,000 |
| dehydrogenase (GAPDH) (60004-1-Ig) | | |
| Phospho-JNK (sc-6254) and Total JNK | Santa Cruz Biotechnology, Texas, | 1:300 |
| (sc-7345) | USA | |
| Phospho-ERK (sc-7383) and Total ERK | Santa Cruz Biotechnology, Texas, | 1:300 |
| (s-514302) | USA | |
| CD9 (sc-13118) | Santa Cruz Biotechnology, Texas, | 1:300 |
| | USA | |
| Calnexin (ab133615) | Abcam, Cambridge, UK | 1:1,000 |
| CD9 (flow cytometry) (48-0091-82) | Life Technologies, USA | 1:20 |
| CD81 (flow cytometry) (MA528658) | Life Technologies, USA | 1:20 |

### Reactive Oxygen Species Measurements

For the determination of ROS in cells directly exposed to PEMFs, cells were seeded in black-walled clear bottom 96-well plates (Costar®) at a density of 3,000 or 6,000 cells per well with 8 replicates per condition. 24 h post-seeding, the cells were rinsed twice with warm phenol-free and FBS-free (PFSF) DMEM (GIBCO) and incubated with 5 UM of CM-H2DCFDA (Invitrogen) in PFSF DMEM for 30 min. The individual 96-well plates were exposed to PEMFs at the indicated direction for 10 min and left in the standard culture incubator for 10 min. The media containing CM-H2DCFDA was washout using PSFS DMEM once before proceeding to ROS measurement using a Cytation™ 5 microplate reader (BioTek) at Ex/EM: 492/520 nm every hour for up to 4 h.

For the determination of ROS in cells treated with conditioned media, the conditioned media was collected from donor suspension cells 30 min post PEMF exposure in the field direction as indicated. Pre-plated cells in 96-well plates were incubated with conditioned media for 2 h before they were then rinsed twice with PSFS DMEM and incubated with CM-H2DCFDA. The subsequent steps were carried out according to the protocol as described above.

### Myotube Differentiation, Fusion Index Quantification and Confocal Microscopy

Myotubes were generated using C2C12 myoblasts seeded at 8,000 cells/cm² in DMEM supplemented with 10% FBS. Differentiation media (DMEM containing 2% horse serum) was added to high-density day 4 and day 6 myoblast cultures. The myotubes will be ready on culture day 8 (Yap, J. L. Y. et al., Faseb j 33, 12853-12872 (2019)). To investigate the effect of C2C12 pCM in suspension on recipient cells, 5× cells were resuspended in 11 ml of basal DMEM in conical tubes. After PEMF exposures, the conical tubes were incubated at 37° C. in a standard tissue culture incubator for 1 h. The cell suspensions were then centrifuged at 1,200 rpm for 5 min and the supernatant was removed and added to growing myotube cultures (day 4 of culture). 9.8 ml of the pCM was supplemented with 200 μl of horse serum to a final concentration of 2%. This step was repeated on day 6 of culture. Hematoxylin and Eosin staining was performed on the day 8 myotubes on coverslips for the quantification of fusion index (as described in Yap, J. L. Y. et al., Faseb j 33, 12853-12872 (2019)). Immunofluorescence staining was done according to the previously described protocol (Tai, Y. K. et al., Front Oncol 11, 783803 (2021)). Briefly, myotubes grown on glass coverslips were permeabilized, blocked and stained with myogenin antibody overnight. The cells were subsequently stained with anti-mouse Alexa Fluor™ 594 antibody (1:1,000; Thermo Fisher Scientific, USA) for 1 h followed by DAPI staining (1 mg/ml; Sigma Aldrich, USA) for 1 min. The stained cells were mounted onto glass slides with Vectashield® Antifade Mounting Medium (Vector Laboratories). Confocal imaging was done using an Olympus FV1000. The myogenin nuclear/cytoplasmic intensity ratio was determined using ImageJ line plot profile function software by dividing the average myogenin nuclear intensity to the cytoplasmic intensity of myotubes.

### Multiplexed Proteomic Analysis of C2C12-Derived Secretome

Conditioned media from C2C12 cells in suspension (375,000 cells/ml) was collected 1 h after a 10-min PEMF exposure by centrifugation at 1,200 rpm for 5 min. The secretome was analyzed using a mouse-specific Myokine Magnetic Bead Panel (Milliplex® Map Kit; MMYOMAG-74K, Merck Millipore, USA) to simultaneously quantify the presence of the following Erythropoietin growth analytes: (EPO), Fibroblast factor (FGF21), Fractalkine/CX3CL1, Follistatin-like Protein 1 (FSTL-1), Interleukin 6 (IL-6), Interleukin 15 (IL-15), Irisin, Leukemia Inhibitory Factor (LIF), Myostatin (MSTN)/GDF8, Oncostatin M (OSM), Osteocrin/Muscurin (OSTN), Osteonectin (SPARC) and Brain-Derived Neurotrophic Factor (BDNF). The immunoassay procedure was performed according to the manufacturer's workflow and subsequently analyzed on the Luminex 200 System (Thermo Fisher Scientific, USA).

### Isolation of PEMF-Exposed C2C12-Derived EVs

Prior to PEMF exposure, C2C12 cells in suspension were given fresh DMEM supplemented with exosome-depleted FBS (5%). After the indicated PEMF exposures, the cell suspensions were then incubated in a 37° C. incubator for 60 min. Thereafter, the conditioned media from C2C12 suspension cells were centrifuged at 1,200 rpm for 5 min followed by centrifugation at 10,000 g for 30 min to remove microvesicles. The supernatant was subsequently ultra-centrifuged at 120,000 g for 2 h using Quick-Seal Round-Top ultracentrifuge tubes on Optima XPN-100 Ultracentrifuge (Beckman Coulter, USA) at 4° C. to isolate EVs. EVs were resuspended in 300 μl of basal DMEM; 100 μl of the exosome suspension was given to each of three technical replicates of pre-plated C2C12 myoblasts in 2 ml of fresh growth media. The corresponding exosome-depleted conditioned media were similarly given to pre-seeded cells. Recipient cells were allowed to grow for 24 h before cell enumeration using the Trypan Blue assay.

### Determination of Myoblast Survival Following the Addition pCM Derive EVs

Exosomes were isolated using the ultracentrifugation method as described above. Myoblast cell suspensions were exposed to magnetic fields with amplitude of 1) 0 mT, 2) 1.5 mT in the downward direction and 3) 1.5 mT in the upward direction. After ultracentrifugation, the exosomes were reconstituted in DMEM-only media and provided to pre-plated myoblasts. As controls, the growth of EVs-supplemented cells were compared to cells grown in DMEM-only or media supplemented with 10% FBS. Cell enumeration using the Trypan Blue exclusion assay was performed 24 h post EV provision.

### The Potency of Fresh and Stored EVs

The potency of exosomes stored away at 4° C. for 7 days in PBS was compared with those freshly harvested (within an hour of resuspension). EV isolation was performed using the ultracentrifugation method as described above. After 7 days of storage, the exosomes were reconstituted in DMEM-only media. EV recipient cells were allowed to grow for 24 h before cell enumeration using the Trypan Blue exclusion assay.

### Determination of Cell Response Following Different Conditioning Times Prior to EV Isolation

Exosomes were collected using the ultracentrifugation method described above. The incubation time for the pCM post-PEMF exposure was varied to include 30 min, 60 min, 90 min and 120 min of conditioning. The exosomes collected after ultracentrifugation were added to recipient cells. Recipient cells were allowed to grow for 24 h before cell enumeration using the Trypan Blue exclusion assay.

### Protein Characterization of C2C12-Derived EVs Using Western Analysis and Flow Cytometry

Isolation of EVs from C2C12 myoblasts in suspension was performed using the ultracentrifugation method as described above. The exosome pellets were either resuspended in 200 μl RIPA buffer or 50 μl PBS containing 0.2% BSA for western analysis or flow cytometry respectively. For western analysis, an equal volume of samples in 4× loading buffer was resolved using 12% SDS-PAGE gel to determine the expression of exosomes in the EV fraction and cytoplasmic proteins in the whole cell lysate. For flow cytometry, samples were stained with fluorescent-labelled CD81 and CD9 at room temperature for 1 h in the dark before a dilution of 200× in PBS for analysis using Cytoflex S Flow Cytometer (Beckman Coulter).

### Physical Characterization of C2C12-Derived EVs Using TEM and NTA

EVs were characterized for morphology and size distribution as previously described (Tong, L., Theranostics 11, 8570-8586 (2021), incorporated herein by reference). In brief, to visualize the morphology, EVs were incubated on formvar film-coated copper grids (FF200-Cu, 200 mesh) for 10 min. Afterward, negative staining of EVs was performed by incubating the grids with 2.5% gadolinium triacetate for 2 min. Images were taken under an FEI TECNAI™ SPIRIT G2 transmission electron microscope (FEI Company, USA). To determine the size distribution and concentrations of EVs, samples were diluted in MilliQ water to 112 μg/mL followed by nanoparticle tracking analysis (NTA) using a NanoSight® NS300 (Malvern Instruments, Malvern, UK). All acquisitions were processed at a camera level setting of 12 or 13, and five videos were recorded for each sample.

### Assessment of Anticancer Properties of pCM Preconditioned Myotubes on 67NR Breast Cancer Cell Survival

Preconditioning of myotubes with myoblast pCM: Myoblast cultures ready for differentiation induction i.e., 3 days post high-density myoblast seeding (day 0) at 75,000 cells/cm² were provided with myoblast cCM or pCM (myoblast cell suspension paradigm; 1 h CM conditioning post PEMF exposure) in the downward or upward directions. The myoblast cCM or pCM were supplemented with 2% horse serum before they were given to the differentiating myoblasts to form myotubes. The myotube cultures received another dose of myoblast cCM or pCM (supplemented with 2% horse serum) on day 5. On day 7, the preconditioned myotubes received a fresh DMEM-only media change before they were magnetically stimulated with 1.5 mT PEMFs, or not, and harvested for CM 6 h later. The pCM from myotubes were given to 67NR breast cancer cells that were seeded 24 h prior. Briefly, 67NR cells were seeded at 30,000 cells/well in a 6-well format and allowed to settle for 24 h. After the provision of myotube pCM, cell viability assessment was carried out using Trypan Blue Exclusion Assay. The reduction in 67NR breast cancer cell survival is a direct reflection of the anticancer potency of the harvested pCM and would be the inverse relationship if provisioned to healthy muscle cells; increase instead of decreased cell number in an opposing trend.

### Statistical Analysis

All statistics were carried out using GraphPad Prism (Version 9) software. One-way analysis of variance (ANOVA) was used to compare the values between two or more groups supported by multiple comparisons.

### Example 2: Brief PEMF Exposure Stimulates Secretome Release

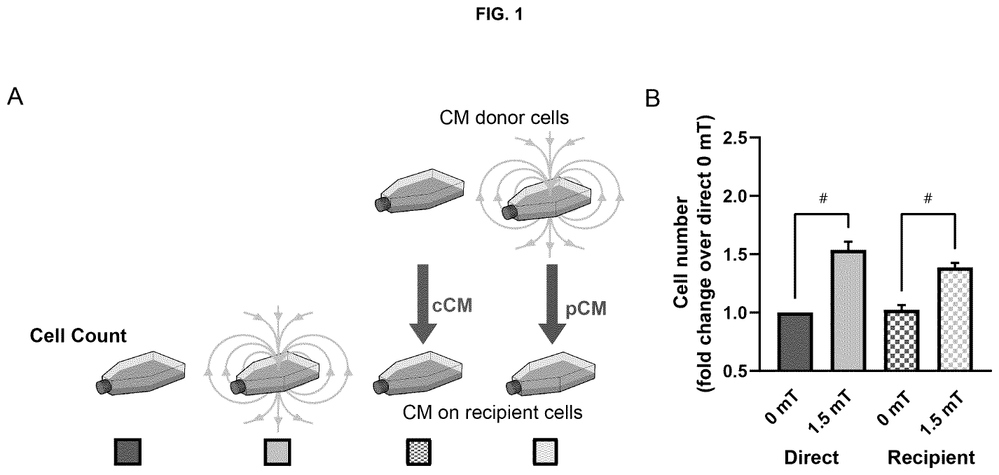

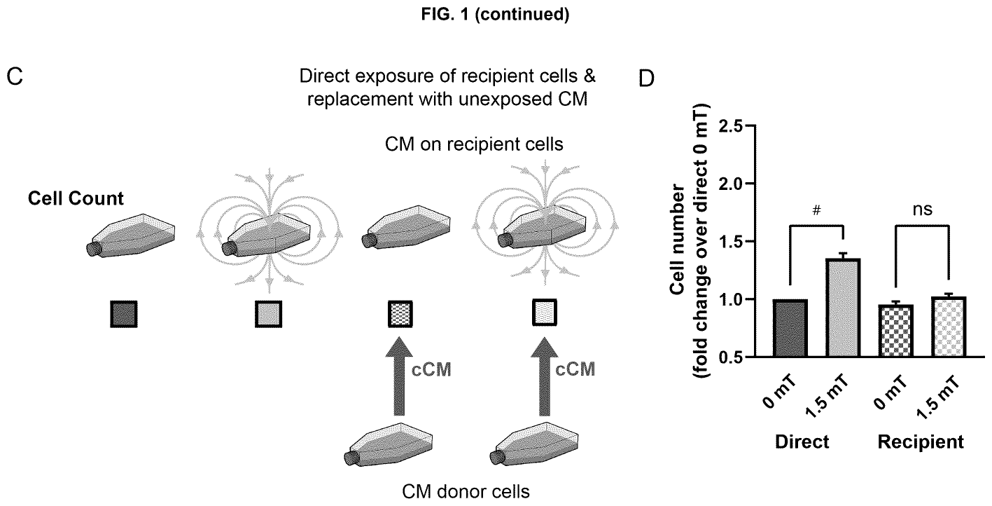

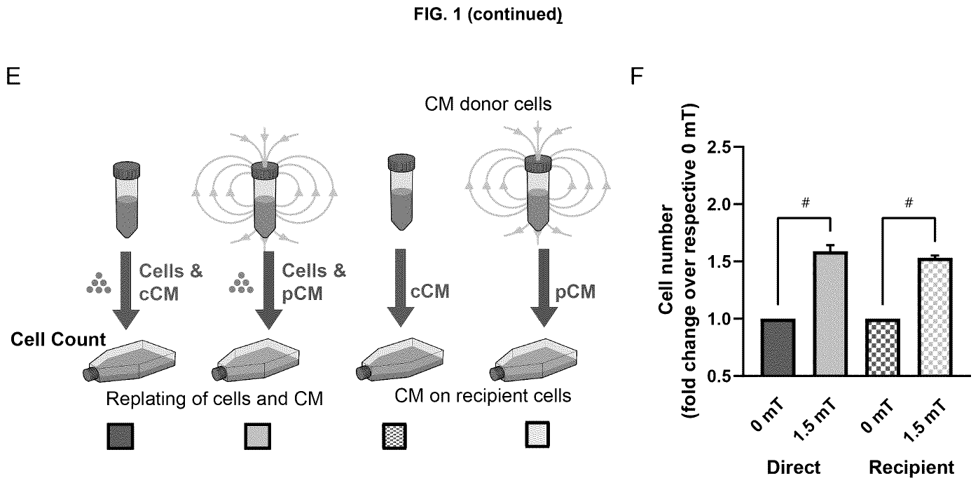

As a standard protocol used for all subsequent experiments, conditioned media was collected from myoblasts after a single 10-min exposure to 1.5 mT amplitude PEMFs or sham treatment (0 mT). To elucidate potential magnetically-induced paracrine effects as previously demonstrated in primary human mesenchymal stem cells (Parate, D. et al., Stem Cell Res Ther 11, 46 (2020)), naïve C2C12 myoblast cultures (recipients) were provisioned with PEMF-conditioned media (pCM) collected from PEMF-stimulated C2C12 cells (donors) and the resulting proliferative responses compared to that of directly exposed or unexposed cells (FIG. 1A). In response to pCM administration, recipient myoblasts exhibited a significant increase in proliferation of similar magnitude to that observed for directly exposed myoblasts (FIG. 1B; light grey), compared to cells provided control conditioned media (cCM) or unexposed cells (FIG. 1B; dark grey), respectively, that exhibited similar levels of growth.

By contrast, removal of the bathing media from C2C12 cultures one hour after PEMF exposure and replacing it with media harvested from age-matched naïve sister cultures (cCM) precluded the typical proliferative response induced by PEMF exposure (FIG. 1C), indicating an underlying paracrine basis for the effect that moreover, appears to be enacted en masse as a single replacement of media one hour after PEMF exposure was able to annul the response.

Aligning with previous studies demonstrating TRPC1-mediated PEMF-responses in suspensions of intact cells (Yap, J. L. Y. et al., Faseb j 33, 12853-12872 (2019); Kurth, F. et al., Biosyst 4, e2000146)) or cell-derived vesicles (Kurth, F. et al., Biosyst 4, e2000146)), it is shown that myoblasts in suspension (FIG. 1E) similarly responded to PEMF exposure with the production of a pCM capable of conferring proliferation enhancement to naïve myoblasts (FIG. 1F) that was similar in magnitude to the proliferative response observed when directly plating suspended cells onto tissue culture plastic after PEMF exposure (FIG. 1F). Notably, the effective conditioning of the pCM from cell suspensions only 15-30 min after PEMF exposure indicates that de novo biosynthesis was not required for the response and rather that the secretome pool was readily poised and immediately released upon magnetic stimulation. Moreover, responses generated from cells in suspension discount substrate-based mechanotransduction as being the underlying force instigating secretome release (Dasgupta, I., and McCollum, D.; J Biol Chem 294, 17693-17706 (2019)). The ability of PEMFs to stimulate the muscle cell secretome hence appears to be a predominantly magnetic phenomenon and, by association, should be subject to modulation by parameters such as a simple change in field orientation (Polk, C.; Journal of Biological Physics 14, 3-8 (1986)).

### Example 3: Magnetic Field Directionality

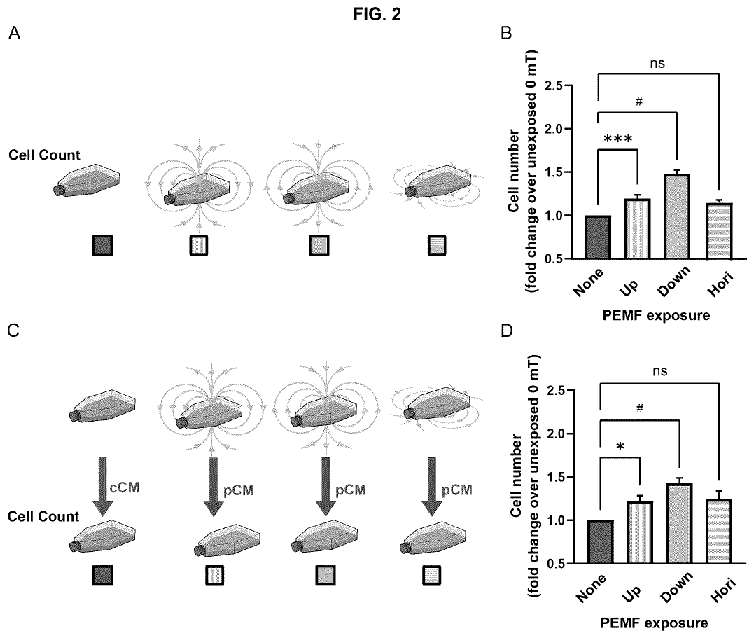

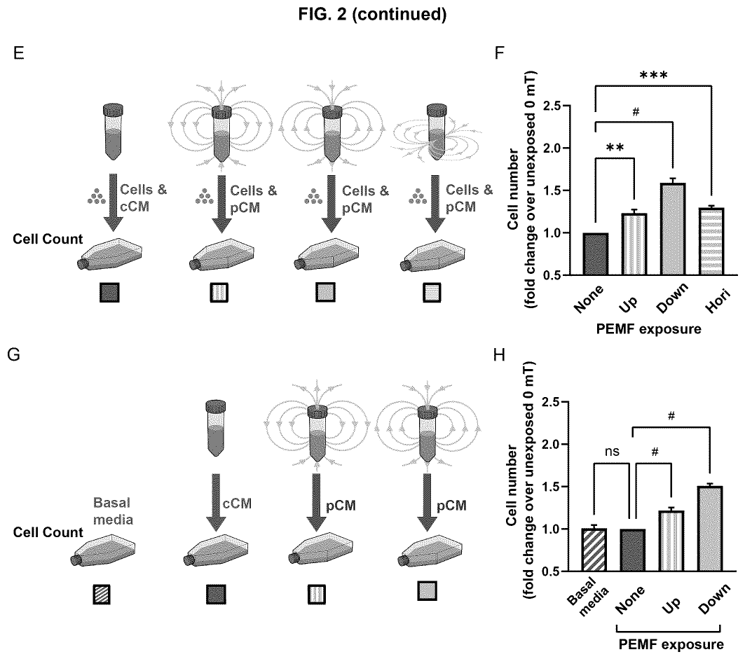

Magnetic field orientation was previously shown to influence MSC-chondrogenesis (Celik, C. et al.; Acta Biomater 119, 169-183 (2021)). In prior experiments (FIGS. 1A-D), culture flasks were positioned horizontally and exposed to PEMFs whose field lines flowed in the downward direction, perpendicular to the cell's long axis. Direct exposure of myoblast cultures in the downward direction (FIG. 2A, solid light grey) rendered a 50% increase in myoblasts proliferation 24 h after exposure (FIG. 2B). By contrast, changing the direction of the magnetic field from downwards to upwards (FIG. 2A, vertical light grey stripes), reduced the induced proliferation response to 20% over non-exposed control cultures (FIG. 2B, dark grey), and exposing myoblasts to magnetic fields aligned parallel to the dish, or horizontally (FIG. 2A, horizontal light grey stripes), rendered the least amount of cell growth that was statistically indistinguishable from no exposure (FIG. 2B). The proliferative potencies of the magnetically-produced secretomes paralleled those of direct exposure (FIG. 2C), being greatest for pCM produced by downward magnetic field exposure (FIG. 2D, solid light grey) and the least for pCM produced by horizontal exposure (FIG. 2D, horizontal light grey stripes). Magnetic field exposure orthogonal to the major axes of the tissue culture flask (and cells) hence exerts greater proliferative effects, with downward exposure producing the overall strongest response. Finally, these effects appear to be largely mediated via the secretome responses of myoblasts.

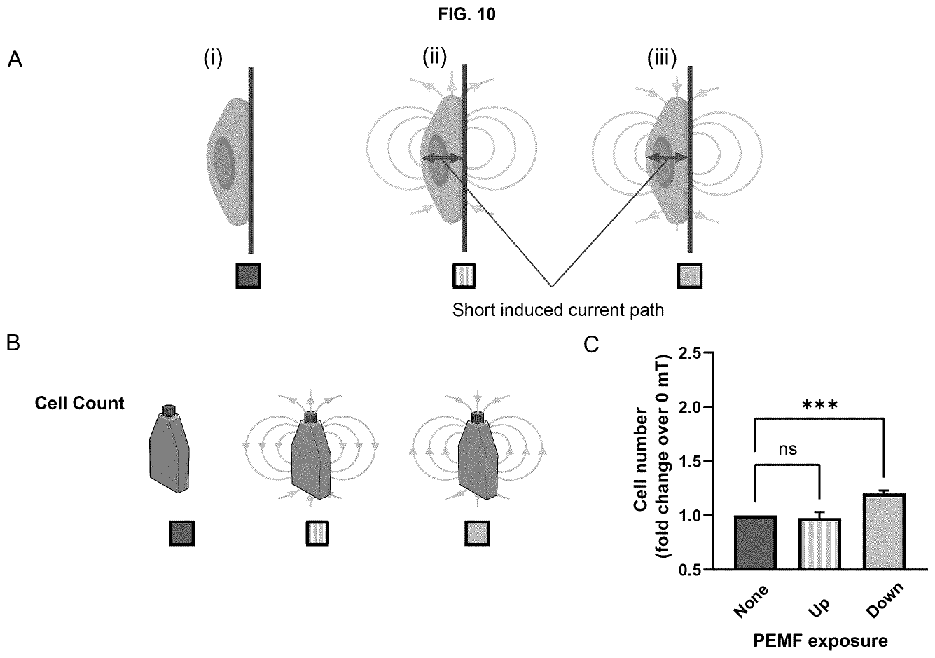

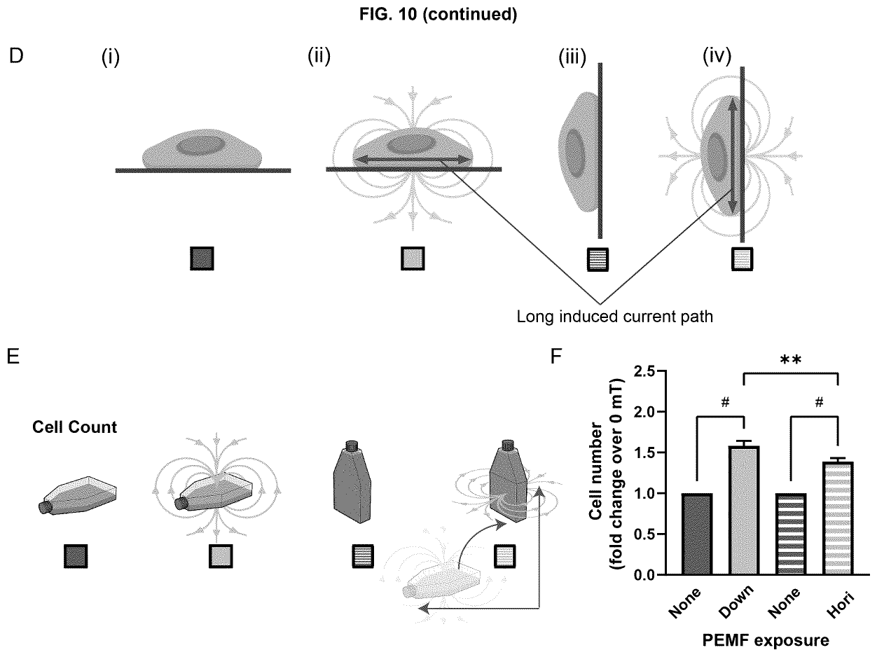

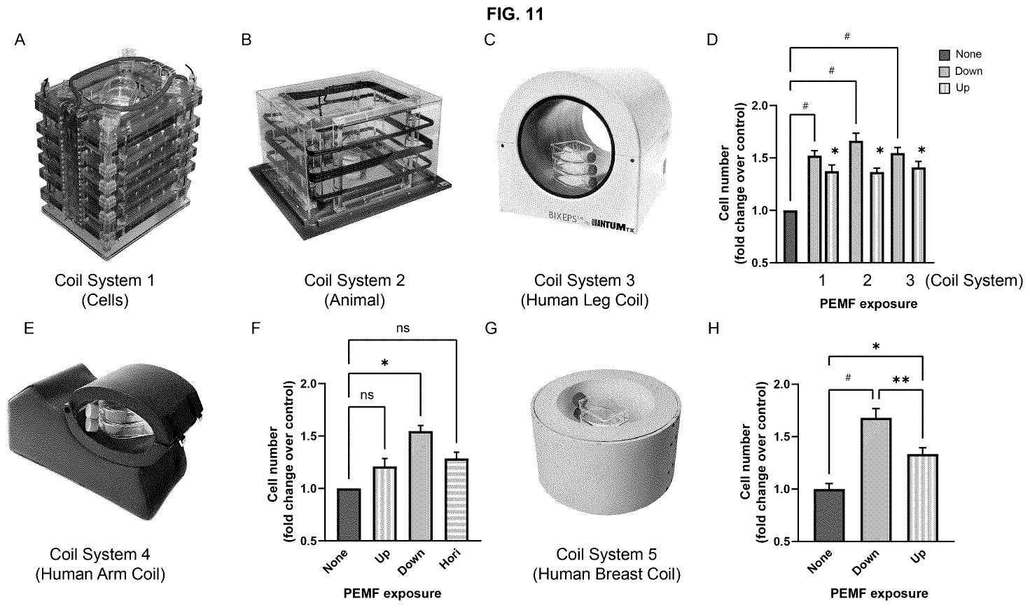

The previous results might suggest that the cell symmetry contributes to magnetotransduction, such that flattened cells on a two-dimensional surface would exhibit long (in-plane of substrate) and short (orthogonal to the plane of substrate) aspects that interact differently with magnetic field direction (FIG. 10). However, if differences in cell symmetry were solely responsible for specificity to magnetic field orientation, then rounded cells in suspension (showing equal aspect ratios) would not be expected to distinguish field orientation via this criterion, much less directionality (up versus down) (FIG. 2E). Provocatively, downward field exposure (FIG. 2F, solid light grey) still produced the greatest proliferative response of cells in suspension of ˜50% over baseline when replated, whereas upward (FIG. 2F, vertical light grey stripes) and horizontal field exposures (FIG. 2F, horizontal light grey stripes) gave nearly identical responses of ˜20% over unexposed controls (FIG. 2F, dark grey). Recapitulating previous results, pCM alone (without cells) harvested from myoblasts exposed to downwardly directed magnetic fields while in suspension (FIG. 2G) produced greater proliferation enhancement (˜50%) of pre-plated recipient myoblasts (FIG. 2H, solid light grey) than pCM harvested from upwardly field exposed myoblasts (˜20%) (FIG. 2H, light grey vertical stripes). By contrast, myoblasts given unexposed cCM (FIG. 2H, solid dark grey) or kept in their basal growth media (FIG. 2H, dark grey hatched) revealed analogous levels of basal proliferation, indicating that the mechanical stimulation (osmotic pressure changes and shear stresses) arising from the media transfer alone is insufficient to stimulate secretome responses. The employed experimental design assured that the secretome levels were not increasing as the result of an inadvertent increase in cell number in response to field exposure, or otherwise. This eventuality was controlled for by the 30-min conditioning of media (too brief to allow for cell division to occur) in the suspension paradigm as well as the 1× cell density condition, reflecting the identical number of cells exposed as in the adherent cultures. Finally, the enhanced proliferation conferred by downward-directed PEMF exposure, relative to upward PEMF-exposure, could be shown to be independent of the device being used (FIG. 11), indicating that technical idiosyncrasies of a particular device are not responsible for the effect as well as demonstrating the feasibility of future translation. Therefore, downwardly directed magnetic fields per se exert proliferative enhancement, independently of cell symmetry and are associated with selective activation of the cell secretome.

### Example 4: Streptomycin Blocks Magnetically-Stimulated Secretome Enhancement

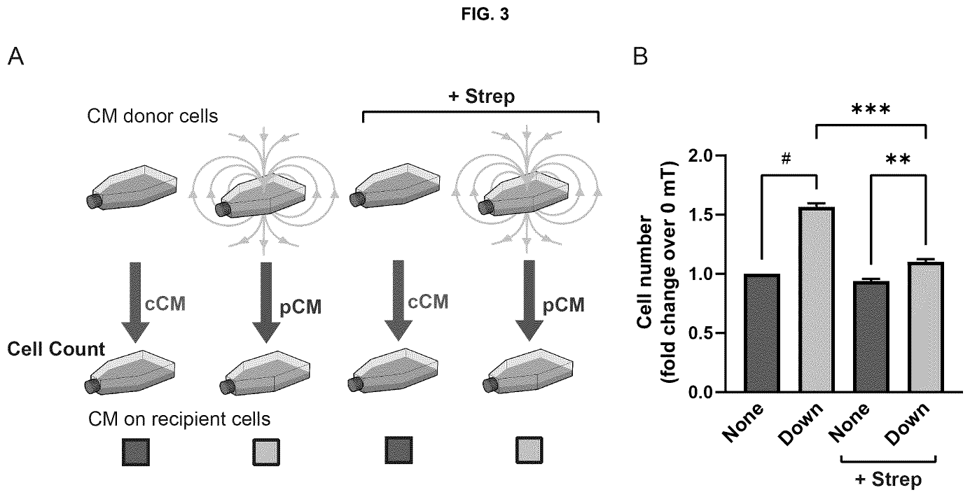

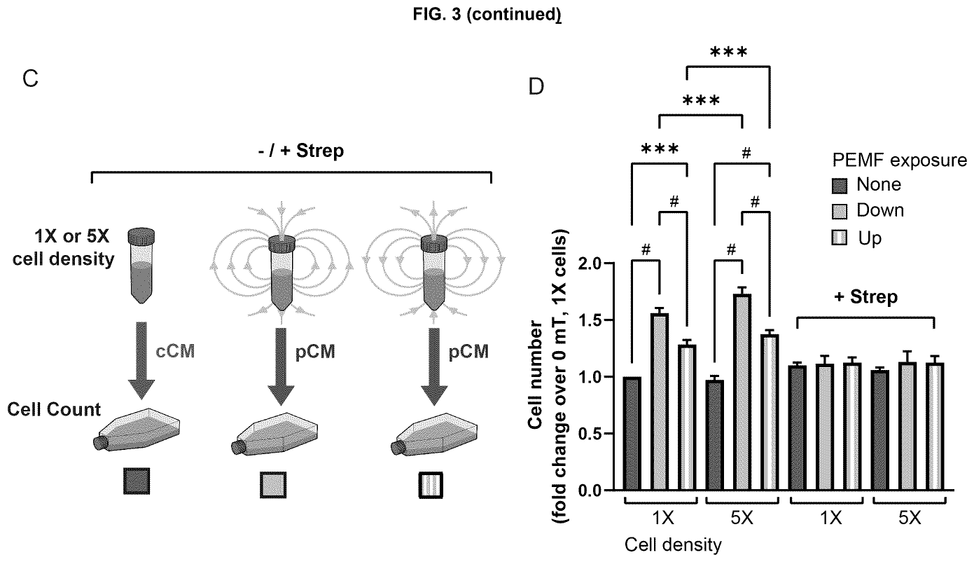

The aminoglycoside antibiotics preclude PEMF-induced myogenic responses by blocking calcium entry via TRPC1 channels, effectively interfering with the activation of NFAT/calcineurin-mediated enzymatic and transcriptional cascades involved in myogenic progression (Yap, J. L. Y. et al., Faseb j 33, 12853-12872 (2019)). Accordingly, streptomycin added to myoblast cultures just before PEMF exposure (FIG. 3A) prevented the production of a myogenic pCM (FIG. 3B). This result reveals the potency of the PEMF-induced secretome compared to constitutive release. Analogously, streptomycin added right before PEMF exposure was also capable of equally preventing the production of a myogenic pCM from myoblasts in suspension at cell concentrations of 15,000/ml (1×) or 75,000/ml (5×) (FIG. 3D). A generalized cytotoxicity of streptomycin cannot underlie the inhibition of proliferation observed in the pCM scenario (light grey; +Strep) as basal proliferation (dark grey) was not reduced by cCMs containing streptomycin (dark grey; +Strep). These combined results underscore the degree by which proliferation is enhanced by factors whose secretion was induced by brief magnetic stimulation.

### Example 5: Direct Downward Field Exposure Preferentially Upregulates Myogenic Proliferation and Differentiation Regulator Protein Expression

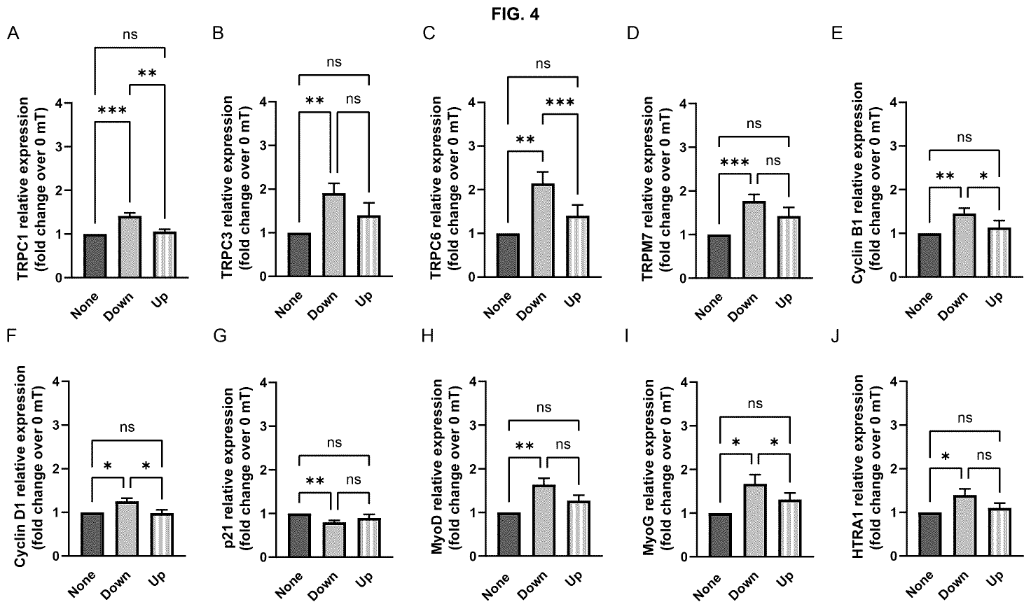

Given previous evidence that analogous magnetic exposure stimulates myogenic proliferation and differentiation in association with TRPC1 function and expression (Yap, J. L. Y. et al., Faseb j 33, 12853-12872 (2019)), it was investigated whether TRPC1 expression was regulated in parallel to secretome response by magnetic field directionality. TRPC1 protein expression in myoblasts was found to be preferentially elevated by exposure to downward PEMFs (FIG. 4A). The protein expression of TRPC1, TRPC3, TRPC6 and TRPM7 were all also preferentially upregulated by downward magnetic fields after 24 h of growth (FIGS. 4A-D). TRPC1 (FIG. 4A) and TRPC6 (FIG. 4C) demonstrated the greatest specificity for field directionality, manifested by the most significant upregulations with downward exposure, relative to upward or no exposure.

Next, the protein expression of regulators of myogenic proliferation and differentiation in response to up or direct downfield exposure was examined. Cyclins B1 (FIG. 4E) and D1 (FIG. 4F) were preferentially upregulated by downward fields, consistent with cell cycle progression in C2C12 muscle cells (Benavides Damm, T. et al., Cell Cycle 12, 3001-3012 (2013)), and in this report associated with proliferation enhancement in response to direct downward field exposure and pCM provision. By contrast, the expression levels of p21, a cyclin-dependent kinase (CDKs) inhibitor (Mansilla, S. F. et al., Genes (Basel) 11 (2020)), were instead reduced by downward magnetic fields and unaltered by upward field exposure (FIG. 4G). Myoblast Determination Protein 1 (MyoD) (FIG. 4H) and Myogenin (MyoG) (FIG. 4I) levels were both preferentially upregulated by downwardly directed magnetic fields consistent with early MyoD and NFAT cotranscriptional regulation of MyoG required for downstream myogenic differentiation (Armand, A. S. et al., J Biol Chem 283, 29004-29010 (2008)), a feature shared with analogous PEMF exposure of myoblasts (Yap, J. L. Y. et al., Faseb j 33, 12853-12872 (2019)) and animals (Tai, Y. K. et al., Faseb j 34, 11143-11167 (2020)). Therefore, both the proliferation and differentiation phases of myogenesis are preferentially enhanced by downward field exposure.

Interestingly, the protein levels of high-temperature requirement A1 (HtrA1) were also significantly elevated by direct downward field exposure (FIG. 4J). HtrA1 is a secreted serine protease and oxidative stress response protein that is reduced in diverse cancers and is reputed to possess tumour-suppressive properties (Chen, M. et al., Cancer Cell Int 21, 513 (2021); Lu, Z. G. et al., Vessel Plus 5 (2021)). Moreover, HTRA1 has been shown to sensitize cancer cells to a broad range of chemotherapies (Chien, J. et al., J Clin Invest 116, 1994-2004 (2006); Folgueira, M. A. et al., Clin Cancer Res 11, 7434-7443 (2005); He, X. et al., Int J Cancer 130, 1029-1035 (2012); Xiong, Z. et al., Ann Clin Lab Sci 47, 264-270 (2017)). It is intriguing to speculate that HTRA1 may belong to the collection of reported exercise-induced myokines with anti-cancer attributes (Bay, M. L., and Pedersen, B. K., Front Physiol 11, 567881 (2020); Kim, J. S. et al., Nat Rev Urol 18, 519-542 (2021); Looijaard, S. et al., Acta Physiol (Oxf) 231, e13516 (2021); Ruiz-Casado, A. et al., Trends Cancer 3, 423-441 (2017)). Muscular secretion of HTRA1 under appropriately administered magnetic stimulation may hence hold important implications for cancer management.

### Example 6: Downward Field Conditioned Media Preferentially Upregulates Myogenic Proliferation and Differentiation Regulator Protein Expression

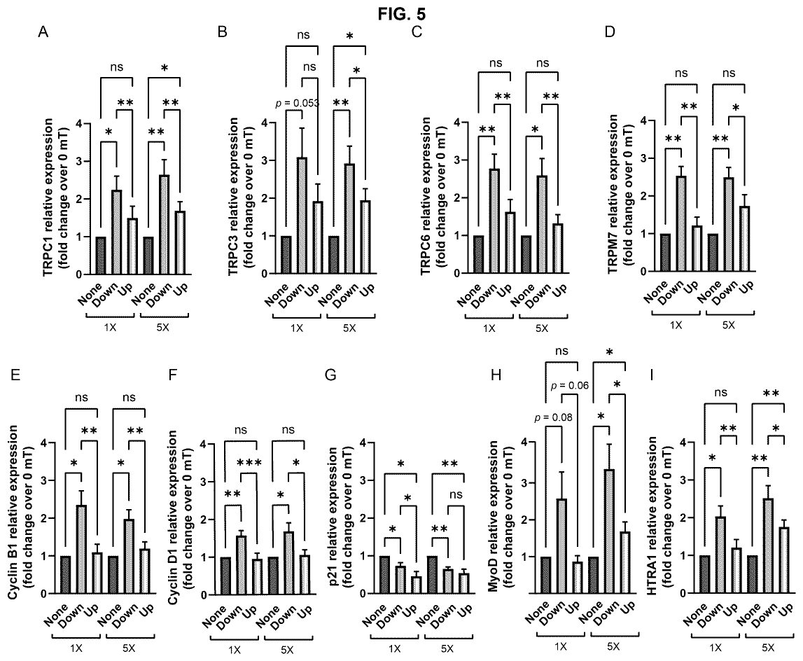

The TRPC channel family exhibits a particular predilection for growth factor regulation of function and expression, expressly TRPC1 with a general outcome of proliferation modulation (Van den Eynde, C. et al., Biochim Biophys Acta Mol Cell Res 1868, 118950 (2021)). Moreover, TRPC1 shows the greatest capacity to heteromultimerize with the other TRPC family members, theoretically uniting their distinct activation modes into a single channel complex (Kiselyov, K., and Patterson, R. L., Front Biosci 14, 45-58 (2009)). To examine whether the upregulation of TRPC channels, cell cycle and myogenic proteins is the cause, or effect, of myokine release, their protein expression levels in recipient cells were examined following incubation in either cCM or pCM generated in response to upward and downward field exposure of myoblasts in suspension at low (1×) and high (5×) cell densities (FIG. 5). Delivery of CM mirrored the effects of direct exposure. Similar to the preferential response of myoblasts to direct downward field exposure, TRPC1 and TRPC6 in recipient myoblasts showed the most selective responses to pCM generated from myoblasts exposed to downward fields. The protein levels of markers of cell cycle and myogenic progression showed a similar preferential upregulation in response to pCM produced by downward field exposure (FIG. 5E-F), whereas p21 protein levels were reduced by pCM of either directionality (FIG. 5G) suggesting a relative attenuation of oxidative stress (Masgras, I. et al., J Biol Chem 287, 9845-9854 (2012)) in response to pCM delivery compared to direct exposure of cells. HTRA1 levels were preferentially upregulated by downward pCM provision (FIG. 51) to a greater degree than direct downward exposure (FIG. 4J). The expressional responses to pCM provision were generally more pronounced than that observed in response to direct exposure of myoblasts (cf. FIG. 4). The immediate release of existing secretome components is reasonably a major contributor to the observed cellular response to magnetic field stimulation and mechanistically aligns with the observed prompt induction of proliferation immediately after PEMF exposure (videos not shown) that is much too rapid to be attributed to the de novo synthesis of secretome elements (Yap, J. L. Y. et al., Faseb j 33, 12853-12872 (2019)).

### Example 7: Direct Exposure to PEMFs Induces ROS Production

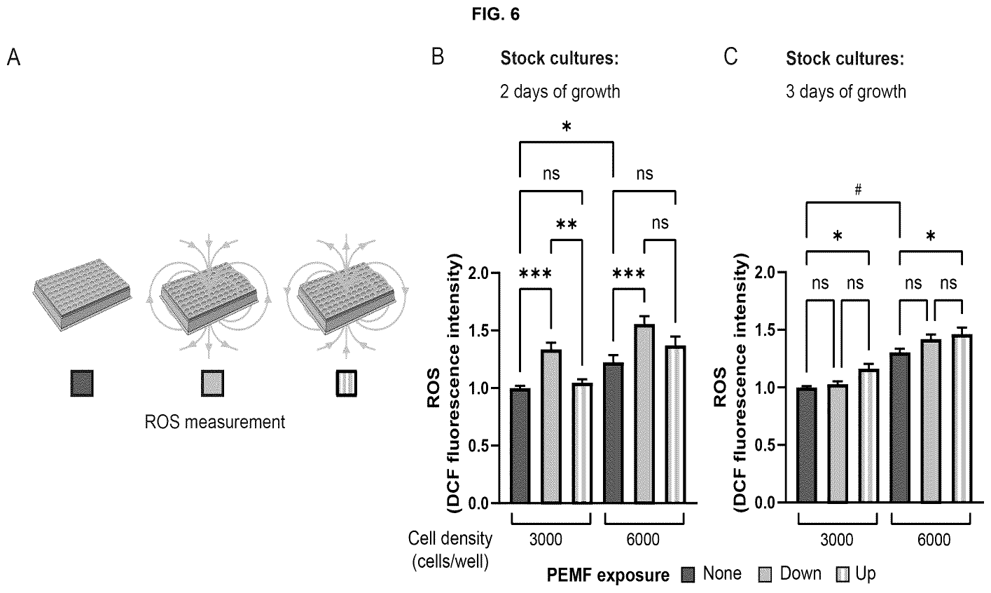

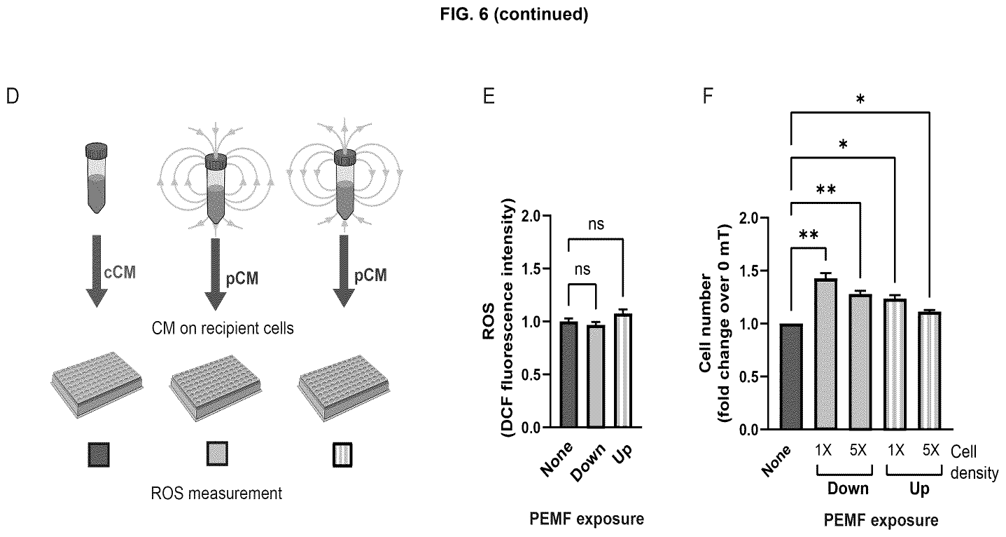

Brief (10 min) exposure to 1.5 mT amplitude PEMFs is capable of inducing a low level of reactive oxygen species (ROS) that promote myoblasts into myogenesis (Yap, J. L. Y. et al., Faseb j 33, 12853-12872 (2019)), whereas longer exposures (1 h) to greater amplitudes (3 mT) PEMFs produces damaging oxidative stress in breast cancer cells (Tai, Y. K. et al., Front Oncol 11, 783803 (2021)). These apparently dichotomous effects are a reflection of the mitohormetic nature of PEMF exposure (Yap, J. L. Y. et al., Faseb j 33, 12853-12872 (2019)), whereby mild PEMF exposure induces low levels of oxidative stress that are adaptive and enhance survival, whereas stronger PEM exposure produces greater levels of oxidative stress that are instead overwhelming to a cell's anti-oxidant defences, stymying their survival (Ristow, M. et al., Dose Response 12, 288-341 (2014)). Therefore, despite mitochondrial ROS production being potentially beneficially adaptive, some level of enzymatic disruption may be expected at a cost of requisite oxidative stress. It was thus investigated whether ROS production is a feature shared by direct exposure to PEMFs and pCM provision (FIG. 6). ROS levels significantly incremented in myoblasts directly exposed to PEMFs (FIGS. 6A-B), but were not apparent in naïve myoblasts provided pCM (FIGS. 6D-E). Aligning with previous results, ROS production was greater following downward field exposure (FIG. 6B). Consistent with previously published results demonstrating that a history of cell overgrowth mitigates sensitivity to magnetic field exposure as a result of TRPC1 downregulation (Yap, J. L. Y. et al., Faseb j 33, 12853-12872 (2019)), the growth of stock cultures for 3 days prior to re-plating for subsequent analysis precluded ROS response to direct magnetic exposure of either direction (FIG. 6C), whereas cells originating from stock cultures grown for only 2 days revealed ROS responses to downward field exposure (FIG. 6B). Furthermore, magnetically-induced ROS production was also smaller from myoblast cultures directly grown at higher density (27%; 6,000 cells/well) than at lower density (34%; 3,000 cells/well) (FIG. 6B). Analogously, plating donor cultures at a 5-fold higher density than normal (1×) reduced the proliferative potency (% 5×-%1×) of the pCM generated by downward or upward exposures by ˜−15% and ˜−13%, respectively (FIG. 6F), despite there being a 5-fold greater number of cells contributing to the secretome response. This effect contrasts with the cell suspension scenario, whereby the pCM generated by downward or upward exposures of high-density (5-fold) cell suspensions enhanced the proliferation of recipient cultures by ˜+24% and ˜+15%, respectively (FIG. 3D). The absence of oxidative stress when pCM is administered alone may underlie its greater myogenic response compared to direct exposure (FIG. 5 vs FIG. 4, respectively). In summary, ROS may not be a necessary evil to benefit from PEMF exposure as associated developmental benefits could be bestowed with secretome administration to unexposed cells or tissues and without the restrictions imposed by contact-inhibition of myoblast responses (Tanaka, K., PLOS One 14, e0222559 (2019)) to magnetic fields (Yap, J. L. Y. et al., Faseb j 33, 12853-12872 (2019)).

### Example 8: Effects of pCM on Myogenic Differentiation and Survival

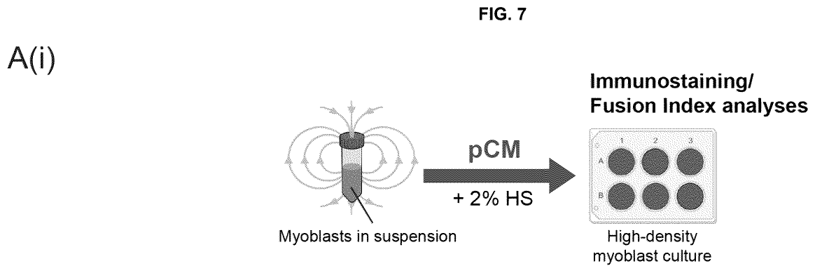

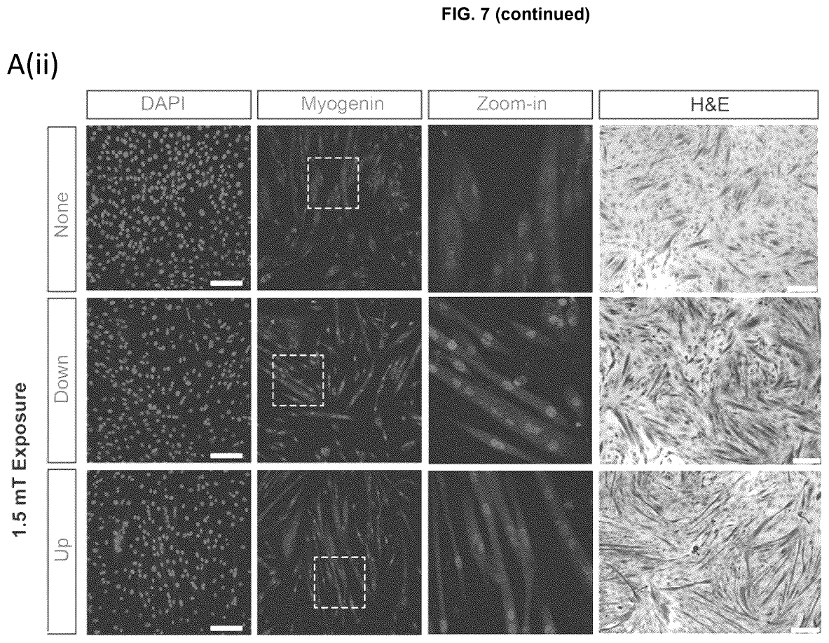

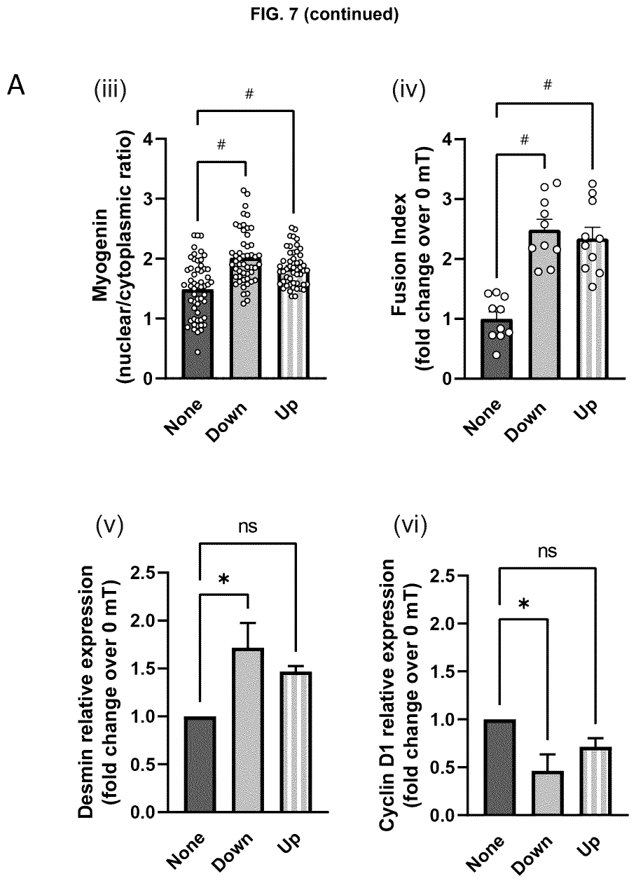

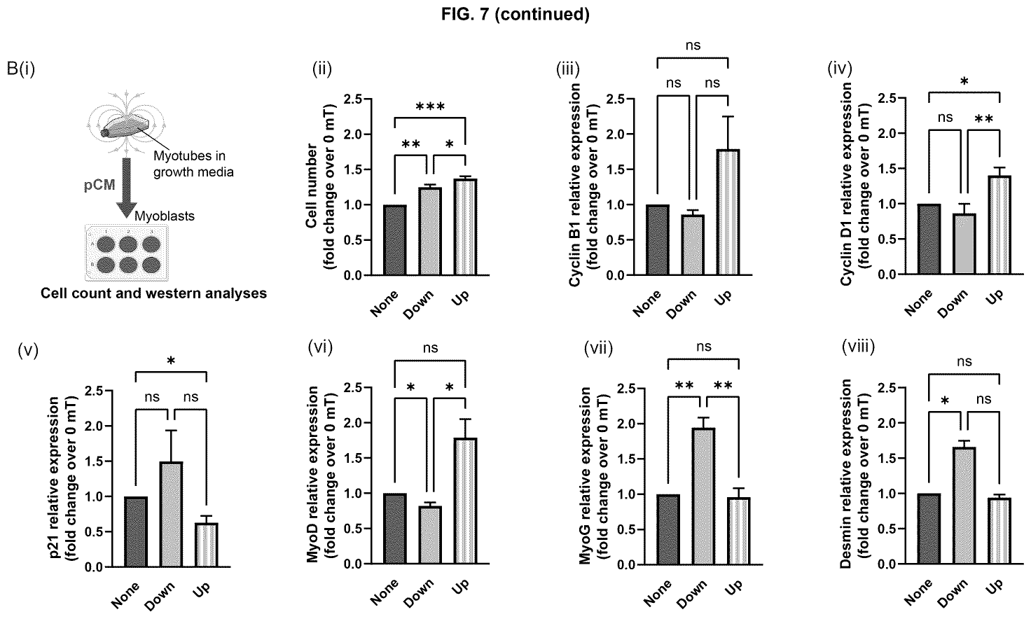

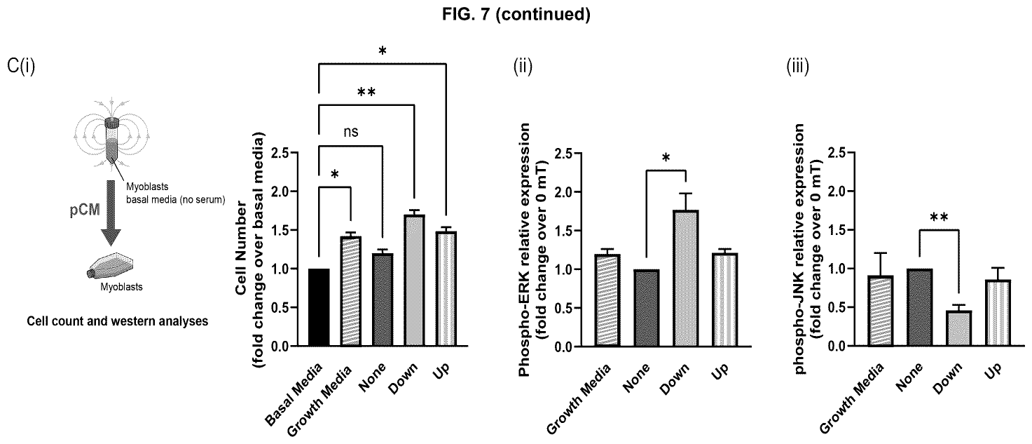

Protein markers of myogenic differentiation were upregulated in myoblast cultures following either direct exposure (FIG. 4) or provision of pCM (FIG. 5) generated in response to downward magnetic fields. Provocatively, the conditioning of base media (DMEM without serum) by myoblasts exposed to downward magnetic fields and then administering to myoblasts in the form of differentiation media (supplemented with 2% Horse Serum) (FIG. 7Ai) stimulated myogenic differentiation (myogenin nuclear translocation, iii; desmin protein expression, v) and myotube formation (fusion index, iv), while slowing proliferation (cyclin D1 protein expression, vi), relative to unconditioned DMEM or DMEM conditioned to upwardly directed magnetic fields (FIG. 7Aii-vi). Notably, despite a greater number of cells (nuclei) in the culture given cCM (none or 0 mT) nuclear myogenin staining was significantly less (FIG. 7Aii). Thus, it appears that conditioned media collected from appropriately magnetically-exposed myoblasts provide secretome components supporting both proliferation (FIG. 5) and differentiation (FIG. 7A), depending on the context of delivery. Conversely, the administration of downward magnetically-conditioned media harvested from differentiated myotubes, promoted the differentiation of proliferating myotubes and forestalled proliferation (FIG. 7B). This result indicates that the contents and attributes of the muscle magnetically-induced secretome is also stage-specific and should be taken into consideration when designing secretome-collecting paradigms. Finally, myoblast conditioning of basal media (minus serum) to downward fields conferred better survival than basal media supplemented with fetal bovine serum (5%) as indicated by dichotomous effects on phospho-ERK and phospho-JNK (54-56) (FIG. 7C) and alluding to the potency of freshly generated secretome.

### Example 9: Validation on Porcine Myoblasts

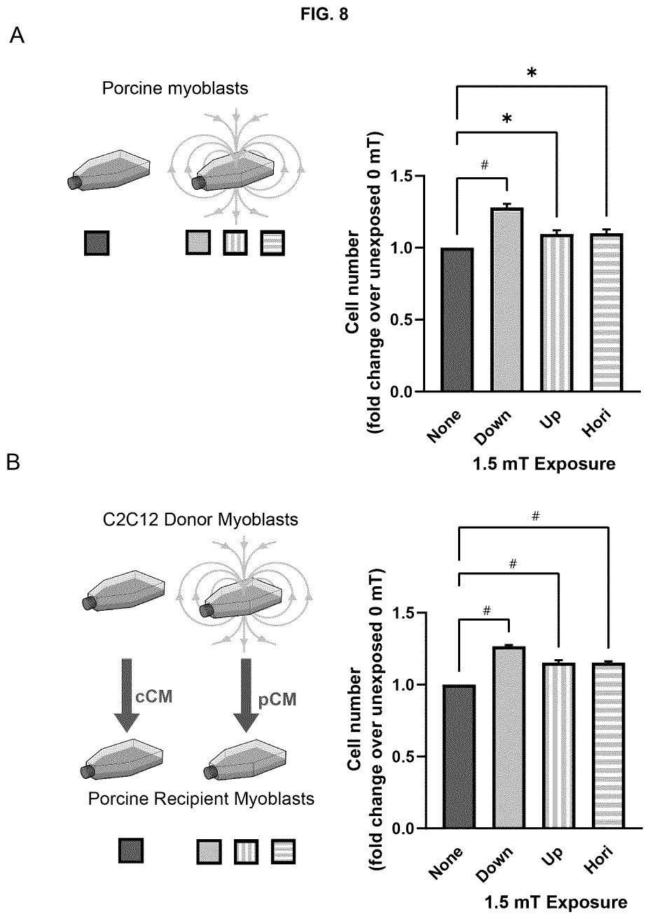

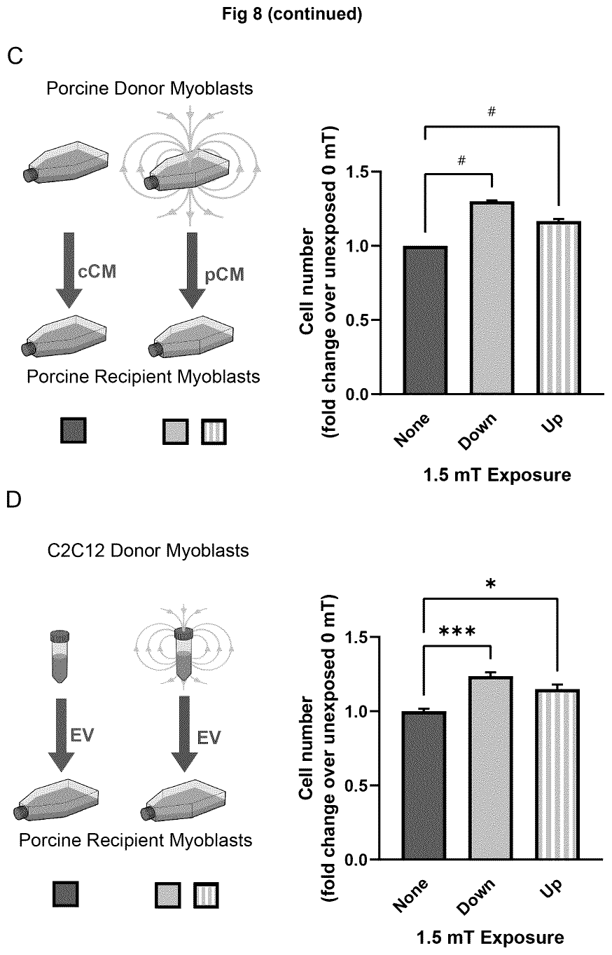

The thus far presented data was generated using the C2C12 muscle murine cell line. To address questions of translatability, the effects of downward magnetic field exposure were validated in an immortalized porcine myoblast cell line. Porcine myoblasts exhibited the characteristic preferential proliferative enhancement to downward magnetic field exposure (FIG. 8A) as well as to pCM generated from either murine C2C12 (FIG. 8B) and porcine (FIG. 8C) myoblasts in response to downward field exposure, demonstrating cross-species efficacy. EVs are an important component of FBS contributing to its myogenic potential across species (Aswad, H., BMC biotechnology 16, 1-12 (2016)). EVs isolated from C2C12 myoblasts were capable of stimulating the proliferation of porcine myoblasts (FIG. 8D) to a similar degree as direct exposure (FIG. 8A) or pCM provision from either C2C12 (FIG. 8B) or porcine (FIG. 8C) myoblasts, indicating they are a major contributor to the observed secretome responses.

### Example 10: Secretome Characterization

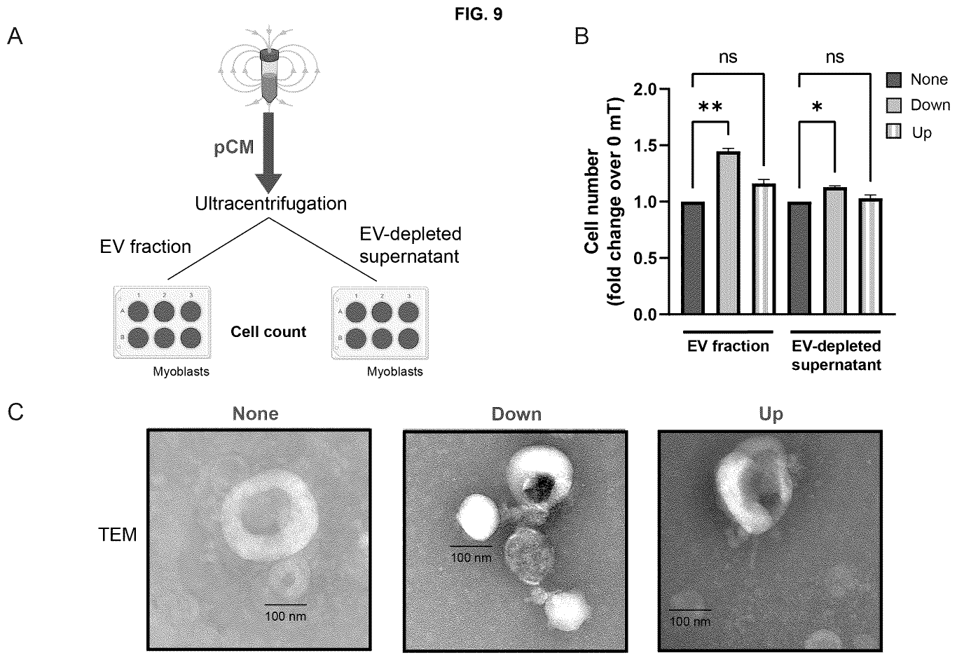

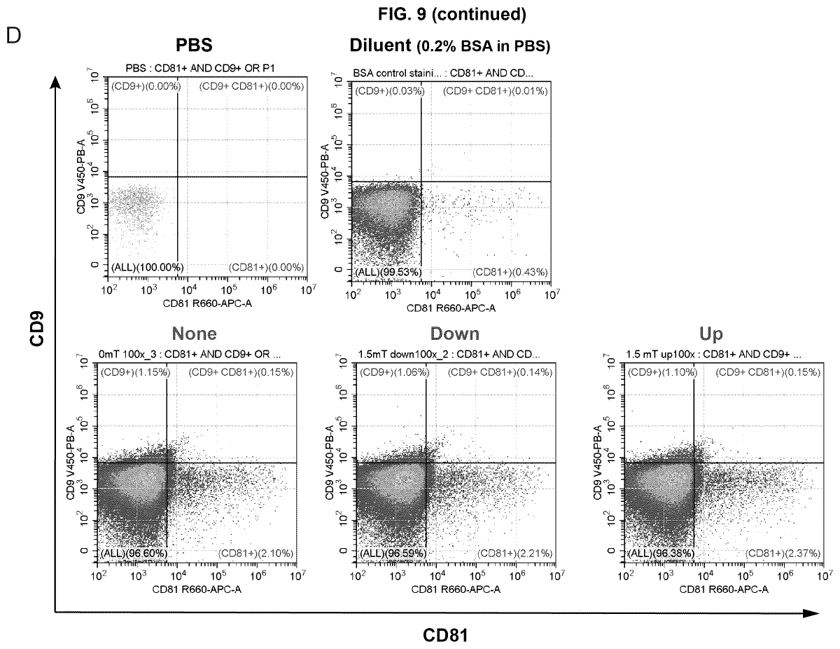

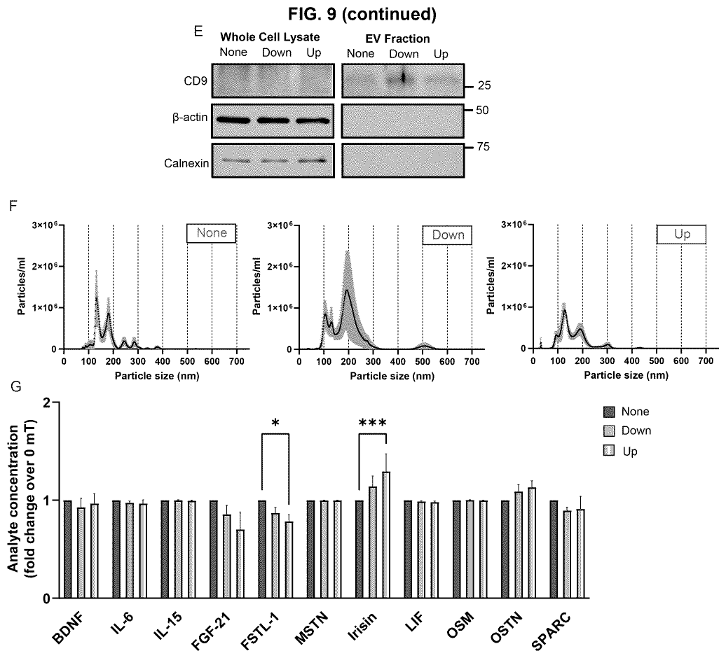

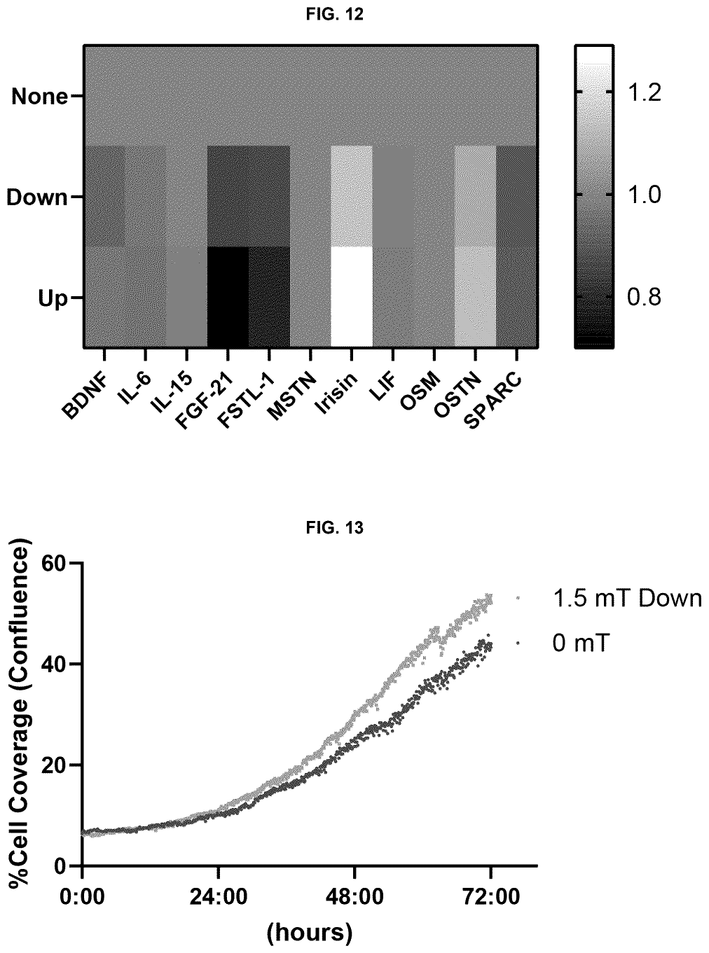

The myogenic potentials of the EV and supernatant fractions of the C2C12 secretome were compared (FIG. 9A). EVs were more capable of promoting proliferation than the supernatant fraction (FIG. 9B), particularly in response to downward-directed magnetic fields (solid light grey). Transmission electron microscopic examination of the vesicular fraction revealed spherical bodies on the order of 100-200 nm in diameters (FIG. 9C). Flow cytometry analysis of the EV fraction revealed the presence (per 500,000 events) of characteristic molecular markers of EVs (FIG. 9D), including the tetraspanin, CD9 and CD81 (Tong, L., Theranostics 11, 8570-8586 (2021)). Despite low levels of detection by flow cytometry, CD9 protein could be detected by Western analysis in the EV fraction, but not supernatant (FIG. 9E). Nanoparticle Tracking Analysis revealed typical particle size distributions in the EV fractions of all three conditioned media scenarios, up, down and no magnetic fields (FIG. 9F). Notably, vesicle diameter tended to increase in the downward field generated sample, whereas particle number tended to decrease in the upward field generated sample. Multiplex immunoassay of myokine expression from the pCM generated from myoblasts in suspension showed nominal changes in soluble myokine release in response to downward magnetic exposure (FIG. 9G; FIG. 12), despite a 24-fold concentration of the pCM from that commonly delivered to the cells. The sum of these results indicates that EVs comprise the predominant component of the magnetically-mobilized secretome generated in the suspension cell paradigm after brief periods (<1 h) of media conditioning.

### Example 11: Efficacy Window in Upwards and Downwards Direction for Myoblasts in Suspension

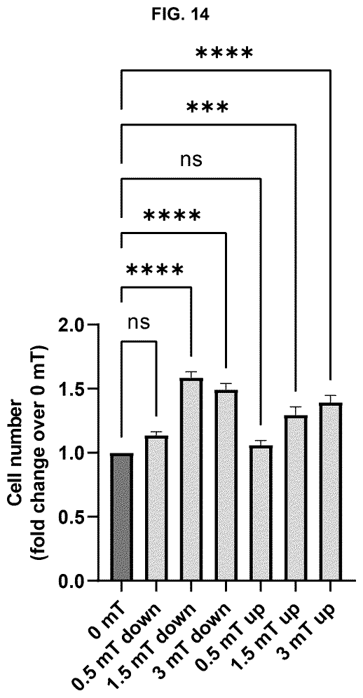

Responses over a range of magnetic field amplitudes were compared. Myoblast proliferation assessment was carried out 24 h following PEMF exposure to myoblasts. It was also found that an efficacy window exists for PEMF exposure where the greatest secretome proliferation effect was produced on myoblasts and myotubes in suspension. When exposed in the downward direction, the peak response was at 1.5 mT. When exposed in the upward direction, there was no evident peak up to an amplitude of 3 mT. However, the response at 3 mT was still smaller compared to exposure at 1.5 mT in the downward direction (FIG. 14).

It was thus found that it is more efficient (less electrical energy required to produce an analogous cell response) to expose cells in the downward direction. Taken together, this data suggests that field directionality is an important determinant in establishing biological efficacy (PEMF efficacy window); in this instance, using less electromagnetic field energy to produce a downward-directed magnetic field for a chosen biological outcome.

### Example 12: Myoblast Survival in Response to EV Fraction of pCM with and without FBS Supplementation

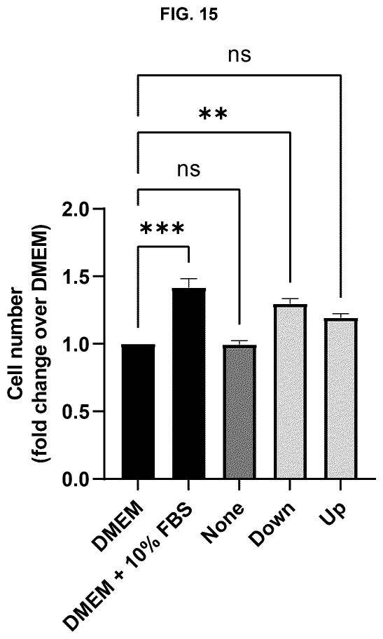

Myoblast proliferation in response to provision of basal media (DMEM without FBS), growth media (DMEM plus 10% FBS) or EVs from myoblast in serum-free DMEM was compared. It was found that isolated exosomes from pCM conferred the same degree of cellular survival as crude pCM when delivered in culture in serum-free media (FIG. 15). This indicates that the EV fraction from pCM is sufficient to confer cellular survival signals and could be used as a replacement for FBS to grow and maintain myoblasts. In the same way as FBS, EVs isolated from pCM from myoblasts or other certain cell types could be used as FBS-replacement for tissue culture.

### Example 13: Influence of Storage and Time of Conditioning Over EV Potency

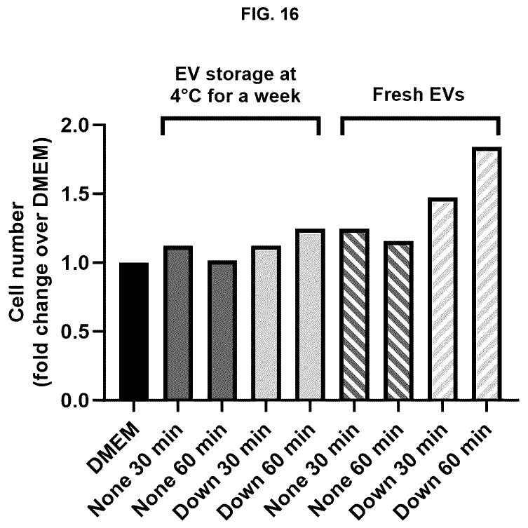

Myoblast proliferation assessment was carried out 24 h following the addition of fresh or one week-old exosomes isolated from exposed myoblasts to naïve recipient myoblasts (n=3 biological replicates with each consisting of 3 technical replicates). It was found that exosomes (EVs) stored at 4° C. in PBS lose their potency within one week. Constitutive release of exosomes decreases after 20-30 min of conditioning, whereas magnetically induced (1.5 mT) exosome release increased and was maximal at 60 min and thereafter it dropped (FIG. 16).

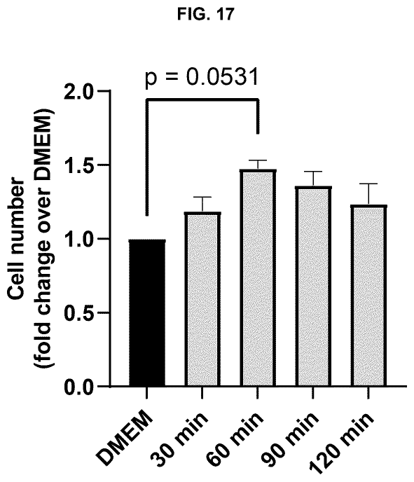

Further, C2C12 myoblasts were exposed to PEMF and incubated for 30 min to 120 min prior to commencing exosome isolation. Myoblast proliferation assessment was carried out 24 h following the addition of exosomes isolated from exposed myoblasts to naïve recipient myoblasts (n=3 biological replicates with each consisting of 3 technical replicates). It was found that the optimal time of conditioning for the isolation of exosomes to induce proliferation in naïve recipient myoblasts was 60 min (FIG. 17).

It was also found that EV size increases with magnetic exposure (FIG. 9F) and exhibits differential EV size generation according to field directionality. Given that EVs contain bioactive components capable of conferring cellular growth, the storage of EVs is an important consideration to maintain their stability and functionality. In addition, the optimal time of conditioning (post-PEMF exposure) was shown to be 60 min. Moreover, the collection of pCM and its associated EVs in 60 min is a crucial consideration to capture the EVs that are docked and available for immediate release from cells. This duration of conditioning ensures the quality and functionality of EVs are a direct reflection of the donor cell developmental (actively proliferating, differentiating cells, etc) and health (healthy, senescent, metabolically inflamed, etc) statuses; 60 min is too brief for the de novo biosnythesis of EVs and for a major reduction in available EVs because of EV reabsorption and utilization by donor cells.

### Example 14: Method to Increase the Anti-Cancer Potency of PEMF-Enhanced Muscle Secretome

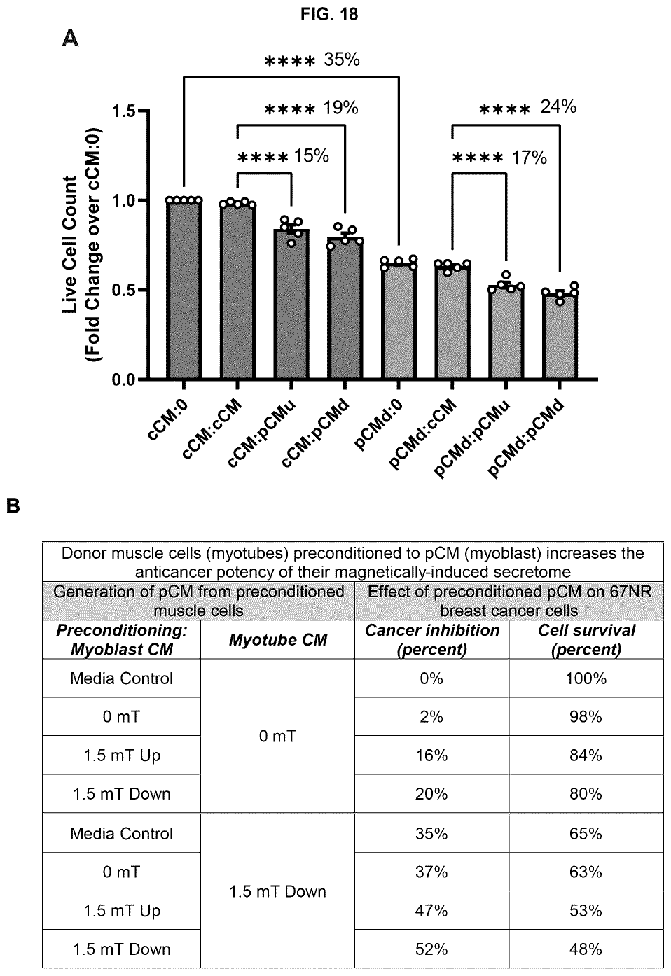

The effect of the administration of the pCM of the present invention on breast cancer (67NR mouse cell line) survival was investigated. Myotubes were preconditioned, or not, with myoblast secretome as indicated (FIG. 18B).

It was found that donor muscle cells (myotubes) preconditioned to pCM (myoblast) increase the anticancer potency of their magnetically-induced secretome (FIG. 18).

In this regard, exposing cells to pCM in their early growth enhances their secretory response upon later exposures (see FIG. 18). Accordingly, a method to produce super secretors (cell pre-conditioning paradigm) may be developed.

Further, the understanding of this paradigm of producing super cell secretors may prove advantageous and can be extended to other cell types for other applications including cell-based meat, cosmetics and wound healing.

### Example 15: Illustrative Application of Invention in Cultivated Meat Production

A major challenge in cell-based meat production is the development of a method of mass-producing culture medium that is myogenic, cost-effective and safe. Traditionally, this objective has been met through supplementation with exogenous growth factors commonly harvested from the foetuses of livestock (Lee, S. Y. et al., J Anim Sci Technol 63, 673-680 (2021); Warner, R. D., Animal 13, 3041-3058 (2019)). Other than being an expensive process, yielding low levels of bioactive factors, this approach also invokes ethical issues surrounding animal cruelty as well as incurs a negative environmental impact. The unmet need was hence a manner to stimulate growth factor release effectively during in vitro meat cultivation with minimal intervention.

Mitochondrial respiration triggers enzymatic cascades that ultimately mobilize secretome release. This important contribution of mitochondrial respiration has been largely ignored in conventional cultured meat paradigms, effectively limiting the quality and quantity of the biomass produced. The PEMF platform presented herein is non-invasive, low-energy, and drug/gene modification-free and is capable of enhancing mitochondrial respiration, myokine release, and myogenesis. A single 10 min exposure of donor muscle cells to correctly oriented PEMFs produces a pCM that after only 30 min of conditioning is capable of enhancing the basal growth of naïve recipient cells by ˜50%, demonstrating the adequacy and potency of the magnetically-induced muscle secretome to promote myogenesis. Indeed, pCM was better able to promote the growth and survival of myoblasts than fetal bovine serum (FIG. 7C). Therefore, in an industrial setting, pCM provision may prove more commercially viable than direct magnetic exposure in enhancing cell-based meat production (compare FIGS. 4 (direct exposure) and 5 (pCM provision)), given the demonstrated low level of oxidative stress associated with direct exposure (FIG. 6).

Cell-to-cell contact-inhibition places restrictions on secretome efficacy (FIG. 6), that would not exist for cells in suspension (FIG. 3C). The rapid and nearly complete release of the muscle cell secretome from cells in suspension also has the potential to optimize operational costs. It has been shown here that 30 min of media conditioning from cells in suspension is sufficient to produce a pCM (FIGS. 3C, 3D) of similar proliferative capacity as pCM harvested from adherent cells after 6 h of exposure (FIGS. 1B, 2D, 3B). Moreover, replacement of the pCM from cells 1 h after exposure with age-matched naïve conditioned media precluded a proliferative response (FIG. 1C), indicating that nearly all of the complete allotment of secretome had been released into the bathing media within an hour of PEMF exposure. Finally, conditioning the media for too long, as to allow cell overgrowth, will produce a secretome with reduced proliferative capacity (FIG. 6F). Magnetic exposure platforms addressing these caveats can be feasibly implemented into existing cell-based meat production pathways with minimal disruption of ongoing production processes, independent of species or ultimate meat objective, and offers key advantages over the state of the art. First, its immediate and highly controllable stimulation of secretome activation from muscle cells will allow for the discovery of secretome components that best support myogenesis. Secondly, it will allow for the scalable production of the secretome from muscle cells in suspension culture, which gains importance in light of the present results showing that cell-to-cell contact may potentially undermine the production of a proliferative secretome (FIG. 6F).

Mechanical stimulation is another manner to stimulate myokine release, but is difficult to achieve in conventional bioreactor paradigms with high acuity and uniformity to all cells. The application of mechanical forces to cells in suspension cultures is lossy and dissipative as freely floating cells largely travel with the flow of the fluid with a minimum of substrate-mediated counterforces that are necessary to produce the shear stresses necessary to instigate secretome release. By contrast, magnetic activation stimulates muscle cells in suspension uniformly and with high temporal acuity and secretome release appears to be very efficient. Moreover, such a magnetic approach would also be clean, humane and commandeer the innate ability of muscles to support their own development with the production of essential growth factors. The ultimate objective would be to reduce the need for exogenous supplementation with animal serum or purified myokines (FIG. 7C) as well as drugs, antibiotics, or genetic modification.

### Example 16: Illustrative Application of Magnetically-Induced Secretome Comprising EVs

The results generated from myoblasts in suspension upon conditioning the media for 30 minutes post-PEMF exposure indicate a predominant contribution of EVs in promoting in vitro myogenesis in this paradigm (FIG. 9). Although an important contribution of EVs from C2C12 myoblasts and fetal bovine serum for the execution of myogenesis has been shown, their role in the cultivated meat industry has not been commonly discussed in the scientific literature (Shaikh, S., Lee, E. et al., Foods 10, 2318. (2021)). The provision of EVs to developing tissues offers practical advantages over the delivery of soluble factors, foremost: 1) their capacity to be conveniently concentrated for storage and delivery as well as; 2) potentially loadable with magnetic exposure. The loading of therapeutic compounds into EVs for drug delivery has proven challenging (de Castilla, P. E. M., Advanced Drug Delivery Reviews 175, 113801 (2021)). The data presented herein would support the notion that appropriately directed magnetic fields may represent a novel and efficient method to load EVs with endogenous therapeutic molecules; EV numbers did not change appreciably upon magnetic exposure, but vesicle size and developmental potency do (FIG. 9F). In this regard, the present invention may provide a technical strategy with demonstrated developmental efficacy.

### SUMMARY

The inventors have successfully shown that the cell secretome can surprisingly and effectively be activated and optimised by a brief exposure to pulsing magnetic fields of a defined directionality (in particular, a downward-direction PEMF exposure as described in the present invention). Given the acknowledged signalling, metabolic and regenerative importance of the muscle secretome, the present invention, which provides a method to rapidly and non-invasively activate its release without the use of drugs or genetic modification has clear scientific, medical and commercial value.

For example, the present invention may be adapted for anti-cancer applications. Induction of cell senescence with change in field direction may provide a secretome with SASP and/or anti-cancer properties which may be used to stall cancer growth. Other clinical applications may include the provision of magnetically-induced secretome comprising healing factors for the treatment of injured tissues. For example, appropriately magnetically-induced dental pulp stem cell secretome may be used to treat injured motor neurons. In another possible example, the present invention may also be adapted for the production of magnetically induced platelet rich plasma (PRP) secretome which has would healing capabilities.

In vitro cell growth commonly requires trophic factor supplementation, either in the form of animal serum or defined factors. In this regard, the inventors have also successfully shown that the pCM of the present invention advantageously has comparable growth and survival capabilities as conventionally employed fetal bovine serum. Therefore, the present invention may provide a medium suitable for supplementation-free (without exogenous and expensive growth factors) cell growth and development.

Further, the present invention as provided herein have been shown to be robust and time-saving, requiring only a short time period to condition the medium to release most of the cells' secretome allotment.

The present invention allows for the production of state-specific secretome and cell type specific secretome. Secretome can be collected from defined cells of interest or at a defined state of development, thus advantageously eliminates secretome contamination from other cell types and from cells in heterogeneous states of development.

The present invention also provides a feeding system with expansion capabilities. Direct exposure of cells is not required to reap the benefits of the secretome. Secretome can be collected from donor cultures (attached or in suspension) for resale or direct provision to cells without the requirement for large-scale implementation of magnetic coil systems for direct exposure of target cells

The present invention also has been shown to have the capacity to selectively collect extracellular vesicles (exosomes and microvesicles) from cells in suspension and may be adapted for enhanced extracellular vesicles production. Conditioned media collected from cells in suspension after 15-30 minutes of exposure is comprised predominantly of the extracellular vesicle fraction, and exosome release is more directionally-sensitive than soluble factor release (myokine, osteokine, adipokine, cytokine, etc.).

As described, the methods of the present invention may be suitable for use in broad applications, such as in regenerative medicine, cosmetic, as well as in the cell-based meat industries.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

Any listing or discussion of an apparently prior-published document in this specification should not necessarily be taken as an acknowledgement that such document is part of the state of the art or is common general knowledge.
Armand, A. S., Bourajjaj, M., Martínez-Martínez, S., el Azzouzi, H., da Costa Martins, P. A., Hatzis, P., Seidler, T., Redondo, J. M., and De Windt, L. J. (2008) Cooperative synergy between NFAT and MyoD regulates myogenin expression and myogenesis. J Biol Chem 283, 29004-29010.
Aswad, H., Jalabert, A., and Rome, S. (2016) Depleting extracellular vesicles from fetal bovine serum alters proliferation and differentiation of skeletal muscle cells in vitro. BMC biotechnology 16, 1-12.
Bay, M. L., and Pedersen, B. K. (2020) Muscle-Organ Crosstalk: Focus on Immunometabolism. Front Physiol 11, 567881.
Benavides Damm, T., Franco-Obregón, A., and Egli, M. (2013) Gravitational force modulates G2/M phase exit in mechanically unloaded myoblasts. Cell Cycle 12, 3001-3012.
Celik, C., Franco-Obregón, A., Lee, E. H., Hui, J. H., and Yang, Z. (2021) Directionalities of magnetic fields and topographic scaffolds synergise to enhance MSC chondrogenesis. Acta Biomater 119, 169-183.
Chen, M., Yang, S., Wu, Y., Zhao, Z., Zhai, X., and Dong, D. (2021) High temperature requirement A1 in cancer: biomarker and therapeutic target. Cancer Cell Int 21, 513.Chien, J., Aletti, G., Baldi, A., Catalano, V., Muretto, P., Keeney, G. L., Kalli, K. R., Staub, J., Ehrmann, M., Cliby, W. A., Lee, Y. K., Bible, K. C., Hartmann, L. C., Kaufmann, S. H., and Shridhar, V. (2006) Serine protease HtrA1 modulates chemotherapy-induced cytotoxicity. J Clin Invest 116, 1994-2004.
Crocetti, S., Beyer, C., Schade, G., Egli, M., Fröhlich, J., and Franco-Obregón, A. (2013) Low intensity and frequency pulsed electromagnetic fields selectively impair breast cancer cell viability. PLOS One 8, e72944.
Dasgupta, I., and McCollum, D. (2019) Control of cellular responses to mechanical cues through YAP/TAZ regulation. J Biol Chem 294, 17693-17706.
de Castilla, P. E. M., Tong, L., Huang, C., Sofias, A. M., Pastorin, G., Chen, X., Storm, G., Schiffelers, R. M., and Wang, J.-W. (2021) Extracellular vesicles as a drug delivery system: A systematic review of preclinical studies. Advanced Drug Delivery Reviews 175, 113801.
Folgueira, M. A., Carraro, D. M., Brentani, H., Patrão, D. F., Barbosa, E. M., Netto, M. M., Caldeira, J. R., Katayama, M. L., Soares, F. A., Oliveira, C. T., Reis, L. F., Kaiano, J. H., Camargo, L. P., Vêncio, R. Z., Snitcovsky, I. M., Makdissi, F. B., e Silva, P. J., Góes, J. C., and Brentani, M. M. (2005) Gene expression profile associated with response to doxorubicin-based therapy in breast cancer. Clin Cancer Res 11, 7434-7443.
Hao, Y., Song, H., Zhou, Z., Chen, X., Li, H., Zhang, Y., Wang, J., Ren, X., and Wang, X. (2021) Promotion or inhibition of extracellular vesicle release: Emerging therapeutic opportunities. Journal of Controlled Release 340, 136-148.
He, X., Khurana, A., Maguire, J. L., Chien, J., and Shridhar, V. (2012) HtrA1 sensitizes ovarian cancer cells to cisplatin-induced cytotoxicity by targeting XIAP for degradation. Int J Cancer 130, 1029-1035.
Kim, J. S., Galvão, D. A., Newton, R. U., Gray, E., and Taaffe, D. R. (2021) Exercise-induced myokines and their effect on prostate cancer. Nat Rev Urol 18, 519-542.
Kiselyov, K., and Patterson, R. L. (2009) The integrative function of TRPC channels. Front Biosci 14, 45-58.
Kurth, F., Tai, Y. K., Parate, D., van Oostrum, M., Schmid, Y. R. F., Toh, S. J., Yap, J. L. Y., Wollscheid, B., Othman, A., Dittrich, P. S., and Franco-Obregón, A. (2020) Cell-Derived Vesicles as TRPC1 Channel Delivery Systems for the Recovery of Cellular Respiratory and Proliferative Capacities. Adv Biosyst 4, e2000146.
Lee, S. Y., Kang, H. J., Lee, D. Y., Kang, J. H., Ramani, S., Park, S., and Hur, S. J. (2021) Principal protocols for the processing of cultured meat. J Anim Sci Technol 63, 673-680.
Li, G., Zhang, L., Wang, D., AlQudsy, L., Jiang, J. X., Xu, H., and Shang, P. (2019) Muscle-bone crosstalk and potential therapies for sarco-osteoporosis. Journal of cellular biochemistry 120, 14262-14273.
Li, J., Li, Y., Atakan, M. M., Kuang, J., Hu, Y., Bishop, D. J., and Yan, X. (2020) The Molecular Adaptive Responses of Skeletal Muscle to High-Intensity Exercise/Training and Hypoxia. Antioxidants (Basel) 9
Looijaard, S., Te Lintel Hekkert, M. L., Wüst, R. C. I., Otten, R. H. J., Meskers, C. G. M., and Maier, A. B. (2021) Pathophysiological mechanisms explaining poor clinical outcome of older cancer patients with low skeletal muscle mass. Acta Physiol (Oxf) 231, e13516.
Louzada, R. A., Bouviere, J., Matta, L. P., Werneck-de-Castro, J. P., Dupuy, C., Carvalho, D. P., and Fortunato, R. S. (2020) Redox Signaling in Widespread Health Benefits of Exercise. Antioxid Redox Signal
Lu, Z. G., May, A., Dinh, B., Lin, V., Su, F., Tran, C., Adivikolanu, H., Ehlen, R., Che, B., Wang, Z. H., Shaw, D. H., Borooah, S., and Shaw, P. X. (2021) The interplay of oxidative stress and ARMS2-HTRA1 genetic risk in neovascular AMD. Vessel Plus 5
Madanagopal, T. T., Tai, Y. K., Lim, S. H., Fong, C. H., Cao, T., Rosa, V., and Franco-Obregón, A. (2021) Pulsed electromagnetic fields synergize with graphene to enhance dental pulp stem cell-derived neurogenesis by selectively targeting TRPC1 channels. Eur Cell Mater 41, 216-232.
Mansilla, S. F., de la Vega, M. B., Calzetta, N. L., Siri, S. O., and Gottifredi, V. (2020) CDK-Independent and PCNA-Dependent Functions of p21 in DNA Replication. Genes (Basel) 11
Masgras, I., Carrera, S., de Verdier, P. J., Brennan, P., Majid, A., Makhtar, W., Tulchinsky, E., Jones, G. D. D., Roninson, I. B., and Macip, S. (2012) Reactive oxygen species and mitochondrial sensitivity to oxidative stress determine induction of cancer cell death by p21. J Biol Chem 287, 9845-9854.
Ost, M., Coleman, V., Kasch, J., and Klaus, S. (2016) Regulation of myokine expression: Role of exercise and cellular stress. Free Radic Biol Med 98, 78-89.
Parate, D., Franco-Obregón, A., Fröhlich, J., Beyer, C., Abbas, A. A., Kamarul, T., Hui, J. H. P., and Yang, Z. (2017) Enhancement of mesenchymal stem cell chondrogenesis with short-term low intensity pulsed electromagnetic fields. Sci Rep 7, 9421.
Parate, D., Kadir, N. D., Celik, C., Lee, E. H., Hui, J. H. P., Franco-Obregón, A., and Yang, Z. (2020) Pulsed electromagnetic fields potentiate the paracrine function of mesenchymal stem cells for cartilage regeneration. Stem Cell Res Ther 11, 46.
Polk, C. (1986) Physical mechanisms by which low-frequency magnetic fields can affect the distribution of counterions on cylindrical biological cell surfaces. Journal of Biological Physics 14, 3-8.
Qin, W., and Dallas, S. L. (2019) Exosomes and extracellular RNA in muscle and bone aging and crosstalk. Current osteoporosis reports 17, 548-559.
Ristow, M., and Schmeisser, K. (2014) Mitohormesis: Promoting Health and Lifespan by Increased Levels of Reactive Oxygen Species (ROS). Dose Response 12, 288-341.
Ruiz-Casado, A., Martín-Ruiz, A., Pérez, L. M., Provencio, M., Fiuza-Luces, C., and Lucia, A. (2017) Exercise and the Hallmarks of Cancer. Trends Cancer 3, 423-441.
Scheele, C., Nielsen, S., and Pedersen, B. K. (2009) ROS and myokines promote muscle adaptation to exercise. Trends Endocrinol Metab 20, 95-99.
Shaikh, S., Lee, E., Ahmad, K., Ahmad, S.-S., Chun, H., Lim, J., Lee, Y., and Choi, I. (2021) Cell Types Used for Cultured Meat Production and the Importance of Myokines. Foods 10, 2318.
Tai, Y. K., Ng, C., Purnamawati, K., Yap, J. L. Y., Yin, J. N., Wong, C., Patel, B. K., Soong, P. L., Pelczar, P., Fröhlich, J., Beyer, C., Fong, C. H. H., Ramanan, S., Casarosa, M., Cerrato, C. P., Foo, Z. L., Pannir Selvan, R. M., Grishina, E., Degirmenci, U., Toh, S. J., Richards, P. J., Mirsaidi, A., Wuertz-Kozak, K., Chong, S. Y., Ferguson, S. J., Aguzzi, A., Monici, M., Sun, L., Drum, C. L., Wang, J. W., and Franco-Obregón, A. (2020) Magnetic fields modulate metabolism and gut microbiome in correlation with Pgc-1a expression: Follow-up to an in vitro magnetic mitohormetic study. Faseb j 34, 11143-11167.
Tong, L., Hao, H., Zhang, Z., Lv, Y., Liang, X., Liu, Q., Liu, T., Gong, P., Zhang, L., Cao, F., Pastorin, G., Lee, C. N., Chen, X., Wang, J. W., and Yi, H. (2021) Milk-derived extracellular vesicles alleviate ulcerative colitis by regulating the gut immunity and reshaping the gut microbiota. Theranostics 11, 8570-8586.
Van den Eynde, C., Vriens, J., and De Clercq, K. (2021) Transient receptor potential channel regulation by growth factors. Biochim Biophys Acta Mol Cell Res 1868, 118950.
Vechetti Jr, I. J., Peck, B. D., Wen, Y., Walton, R. G., Valentino, T. R., Alimov, A. P., Dungan, C. M., Van Pelt, D. W., von Walden, F., and Alkner, B. (2021) Mechanical overload-induced muscle-derived extracellular vesicles promote adipose tissue lipolysis. The FASEB Journal 35, e21644.
Warner, R. D. (2019) Review: Analysis of the process and drivers for cellular meat production. Animal 13, 3041-3058.
Wong C. J. K, Tai Y. K., Yap J. L. Y., Fong C. H. H., Loo L. S. W., Kukumberg M., Fröhlich J., Zhang S., Li J. Z., Wang J. W., Rufaihah A. J., Franco-Obregón A. (2022) Brief exposure to directionally-specific pulsed electromagnetic fields stimulates extracellular vesicle release and is antagonized by streptomycin: A potential regenerative medicine and food industry paradigm. Biomaterials. 2022 Jul. 6; 287:121658. doi: 10.1016/j.biomaterials.2022.121658. Epub ahead of print. PMID: 35841726.
Xiong, Z., Fu, Z., Shi, J., Jiang, X., and Wan, H. (2017) HtrA1 Down-regulation Induces Cisplatin Resistance in Colon Cancer by Increasing XIAP and Activating PI3K/Akt Pathway. Ann Clin Lab Sci 47, 264-270.
Yap, J. L. Y., Tai, Y. K., Frohlich, J., Fong, C. H. H., Yin, J. N., Foo, Z. L., Ramanan, S., Beyer, C., Toh, S. J., Casarosa, M., Bharathy, N., Kala, M. P., Egli, M., Taneja, R., Lee, C. N., and Franco-Obregón, A. (2019) Ambient and supplemental magnetic fields promote myogenesis via a TRPC1-mitochondrial axis: evidence of a magnetic mitohormetic mechanism. Faseb j 33, 12853-12872.

1. A method of producing a conditioned medium capable of promoting proliferation, differentiation or senescence of progenitor and/or stem cells, wherein the method comprises the steps:
a) culturing proliferating, differentiating or senescent (oxidatively stressed) progenitor and/or stem cells in media as a suspension culture in a bioreactor;
b) exposing the proliferating, differentiating, or senescent (oxidatively stressed) progenitor and/or stem cells to downward-directed or upward-directed low amplitude pulsed electromagnetic fields (PEMFs); and
c) collecting the PEMF-conditioned media (pCM) which comprises a secretome that reflects the status of the cells in the bioreactor and has cell proliferation-promoting capability, cell survival-promoting capability, cell differentiation-promoting capability, or cell senescence-promoting capability.

2. The method of claim 1, wherein
bi) the pCM from proliferating progenitor and/or stem cells exposed to downward-directed or upward-directed low amplitude PEMFs promote proliferation, with the exception that pCM from proliferating myoblasts exposed to downward-directed PEMFs will promote differentiation; or
bii) the pCM from differentiating progenitor and/or stem cells exposed to downward-directed low amplitude PEMFs promote differentiation, whereas exposure to upward-directed PEMFs will promote proliferation and/or survival; or
biii) the pCM from senescent (oxidatively stressed) progenitor and/or stem cells exposed to downward-directed or upward-directed low amplitude PEMFs to promote senescence;
wherein upward-directed low amplitude PEMFs are less efficient than downward-directed low amplitude PEMFs at activating mitochondrial oxygen-based respiration and generating reactive oxygen species (ROS) and require administration at higher amplitude than downward-directed low amplitude PEMFs to have a similar effect.

3. The method of claim 1, wherein the progenitor and/or stem cells are exposed to the PEMFs:
a) for a single 10-30 minute duration, and/or
b) at a downward-directed amplitude of 0.5-2 mT for muscle cells, 0.5-2 mT for fibroblast cells, 0.5-3 mT for hematopoietic stem cells, 2.5-3.5 mT for mesenchymal cells, or 1.5-2.5 mT for dental pulp cells, and/or
c) in 20×150 μs on and off pulses for 6 ms at a repetition frequency of 15 to 50 Hz.

4. The method of claim 1,
wherein the progenitor and/or stem cells are in and/or on free-floating micro scaffolds; and/or
wherein the progenitor and/or stem cells are myoblast cells, neuronal stem cells, hematopoietic stem cells, dental pulp stem cells, fibroblast cells or mesenchymal stromal cells; and/or
wherein the progenitor and/or stem cells are differentiated or proliferating.

5. (canceled)

6. The method of claim 1,
wherein the culturing in step a) is performed in the absence of TRPC1 receptor inhibitors, such as aminoglycoside antibiotics; and/or
wherein the culturing in step a) is in serum-free and exogenous growth and/or trophic factor-free media.

7. The method of claim 1, wherein the progenitor and/or stem cells in step a) have been prior expanded and/or conditioned to be in a proliferating, differentiating or senescent state in growth media or media of defined composition.

8. (canceled)

9. A method of proliferating or differentiating progenitor and/or stem cells, comprising adding the pCM from proliferating or differentiating cells, respectively, as defined in claim 1 to a progenitor cell culture.

10. The method of claim 9, wherein the pCM is produced from myoblasts and is used to feed a cell-based meat culture.

11. A system comprising;
i) a bioreactor within which a first culture of progenitor and/or stem cells in suspension is subjected to downward-directed low amplitude pulsed electromagnetic fields (PEMFs), or upward-directed low amplitude pulsed electromagnetic fields (PEMFs), to produce a PEMF-conditioned media (pCM), and
ii) a second cell culture to be expanded (via induced proliferation), differentiated or induced into a senescent (oxidatively stressed) state,
wherein said PEMF-conditioned media (pCM) from (i) is provided to cell culture (ii).

12. (canceled)

13. The system of claim 11, wherein the progenitor and/or stem cells are exposed to the PEMFs:
a) for a single 10 to 30-minute duration, and/or
b) at a downward-directed amplitude of 0.5-2 mT for muscle cells, 0.5-2 mT for fibroblast cells, 0.5-3 mT for hematopoietic stem cells, 2.5-3.5 mT for mesenchymal cells, or 1.5-2.5 mT for dental pulp cells, and/or
c) in 20×150 μs on and off pulses for 6 ms at a repetition frequency of 15 to 50 Hz.

14. The system of claim 11,
wherein the progenitor and/or stem cells are in and/or on free-floating micro scaffolds; and/or
wherein the progenitor and/or stem cells in step i) have been prior expanded and/or conditioned to be in a proliferating, differentiating or senescent (oxidatively stressed) state in a growth media or media of defined composition; and/or
wherein the progenitor and/or stem cells are myoblast cells, neuronal stem cells, hematopoietic stem cells, dental pulp stem cells, fibroblast cells or mesenchymal stromal cells; and/or
wherein the progenitor and/or stem cells are differentiated or proliferating; and/or
wherein the progenitor and/or stem cells in i) are myoblast cells and the cell culture ii) is for cell-based meat production.

15. (canceled)

16. (canceled)

17. (canceled)

18. The system of claim 11, wherein the culturing in step i) is in serum-free and exogenous growth and/or trophic factor-free media.

19. A method of enhancing cultured meat production, comprising feeding a cell-based meat culture a pCM produced by the method of claim 1.

20. The method of claim 19, wherein the cell-based meat culture is fed:
a) a proliferating myoblast-derived pCM to promote proliferation of the cells in said culture, and then
b) a differentiating myoblast-derived, or differentiated myotube-derived, pCM to promote differentiation of the proliferating cells in said culture to form a cell-based meat;
wherein the pCM is produced in a serum-free and exogenous growth and/or trophic factor-free media.

21. (canceled)

22. A pCM, produced by the method of claim 1, preferably comprising exosomes.

23. A method of pre-conditioning proliferating, differentiating or senescent (oxidatively stressed) progenitor and/or stem cells for use in the production of a PEMF-conditioned media (pCM), wherein the method comprises contacting a sample of proliferating, differentiating or senescent (oxidatively stressed) progenitor and/or stem cells with the pCM according to claim 22,
wherein the contacting with the pCM enhances the secretory response of said proliferating, differentiating or senescent (oxidatively stressed) progenitor and/or stem cells upon later exposure to PEME.

24. (canceled)

25. A method of producing a conditioned medium capable of inhibiting the proliferation, migration and/or invasiveness of cancer cells, wherein the method comprises the steps:
a) exposing a suspension culture of the progenitor and/or stem cells to downward-directed low amplitude pulsed electromagnetic fields (PEMFs); and
b) collecting the PEMF-conditioned media (pCM) which comprises a secretome; and
c) adding the collected pCM from step b) to precondition differentiating myoblast cells, or differentiated myotube cells, in culture and:
i) collecting constitutively released secretome (cCM) from resultant myotube cells; or
ii) exposing resultant myotube cells to low amplitude PEMFs and collecting the PEMF-conditioned media (pCM) which comprises a secretome.

26. The method of claim 25, wherein the progenitor and/or stem cells are myoblast cells, and wherein the PEMF exposure is:
a) for a single 10-30 minute duration, and/or
b) at a downward-directed amplitude of 0.5-2 mT, and/or
c) in 20×150 μs on and off pulses for 6 ms at a repetition frequency of 15 to 50 Hz.

27. A cCM or pCM capable of inhibiting the proliferation, migration and/or invasiveness of cancer cells, produced by the method of claim 25.

28. A method of inhibiting the proliferation, migration and/or invasiveness of cancer cells, comprising contacting an efficacious amount of the pCM or cCM from step c) defined in claim 25 with said cancer cells.
